(19) United States
(12) Patent Application Publication
(10) Pub. No.: US 2026/0108488 A1
(43) Pub. Date: Apr. 23, 2026

(54) PROCESS FOR PREPARING BIOCHANIN A-LOADED HYDROGEL FOR PROMOTING WOUND HEALING IN DIABETIC PATIENTS

(71) Applicants: Abeer S. ALGHAMDI, Jeddah (SA); Khadijah B. ALKINANI, Makkah (SA); Ashraf B. ABDEL-NAIM, Jeddah (SA); Imran KAZMI, Jeddah (SA); Fahad A. ALABASSI, Jeddah (SA)

(72) Inventors: Abeer S. ALGHAMDI, Jeddah (SA); Khadijah B. ALKINANI, Makkah (SA); Ashraf B. ABDEL-NAIM, Jeddah (SA); Imran KAZMI, Jeddah (SA); Fahad A. ALABASSI, Jeddah (SA)

(21) Appl. No.: 19/425,665

(22) Filed: Dec. 18, 2025

(51) Int. Cl. A61K 31/352; A61K 9/06; A61K 47/38

(52) U.S. Cl. CPC A61K 31/352; A61K 9/06; A61K 47/38

(57) ABSTRACT

The invention provides a process for preparing a Biochanin A (BCA)-loaded hydroxypropyl methyl cellulose (HPMC) hydrogel aimed at promoting wound healing in diabetic patients. The hydrogel is prepared by dispersing HPMC in distilled water, followed by dissolving BCA into the mixture with continuous stirring, then storing under refrigeration. The therapeutic potential of Biochanin A (BCA) in promoting wound healing, is evaluated in rats induced with diabetes. The results showed that the BCA-loaded hydrogel accelerates diabetic wound repair by boosting antioxidant defenses via upregulating Nrf2 and related enzymes, suppressing inflammatory markers such as IL-1β, IL-6, TNF-α, COX-2, and TLR4, and stimulating angiogenesis through increased expression of TGF-β1, VEGF-A, and PDGFR. It promotes faster wound contraction, re-epithelialization, collagen deposition, granulation tissue formation, and neovascularization, potentially reducing the risk of complications like limb amputation. The invention offers a promising therapeutic approach for treating chronic diabetic wounds with improved healing outcomes.

### FIELD OF THE INVENTION

The present disclosure relates to a process for preparing Biochanin A (BCA)-loaded hydrogel for promoting wound healing in diabetic patients. In more particular manner, the present invention relates to the preparation of BCA-loaded hydrogel, wherein prepared hydrogel is administrated to rats induced with diabetes for evaluating, its efficacy in promoting wound healing in diabetic rats.

### BACKGROUND OF THE INVENTION

Diabetes mellitus (DM) constitutes a disorder of metabolism primarily defined by persistent elevation of blood glucose levels (chronic hyperglycemia). This pathological condition develops through one of two principal mechanisms: either the pancreatic tissue fails to synthesize adequate insulin quantities, or target tissues throughout the body demonstrate resistance to insulin action despite its presence in circulation. Both pathways ultimately manifest as disrupted glucose homeostasis, resulting in the characteristic hyperglycemic state that defines this metabolic disease. This pervasive condition affected an estimated 9.3% of the global population in 2019 (463 million people), with projections indicating a significant increase to 10.2% (578 million) by 2030 and 10.9% (700 million) by 2045, as reported by Saeedi. Notably, urban areas and high-income countries experience higher diabetes prevalence rates (10.8% and 10.4%, respectively) compared to their rural and low-income counterparts (7.2% and 4.0%). Within the Middle East, Saudi Arabia ranks second in diabetes prevalence, with an estimated 7 million individuals diagnosed with diabetes and nearly 3 million with pre-diabetes. Diabetes triggers a cascade of acute complications, including but not limited to cardiovascular diseases, cerebrovascular diseases, renal disorders, and obesity. Furthermore, diabetes significantly impairs the healing of serious wounds, affecting approximately 25% of all diabetic patients and often culminating in lower limb amputation, with substantial economic and psychosocial ramifications.

Wound healing is a multifaceted, dynamic process consisting of distinct, predictable stages: hemostasis (blood clotting), inflammation, cellular proliferation (tissue growth), and tissue remodeling, which involves both maturation and cell specialization. Minor acute wounds typically heal completely in healthy individuals within two to three weeks. Conversely, diabetic wounds can progress to a chronic state, failing to heal within six to eight weeks, due to an imbalance in the body's natural healing process. Moreover, bacterial colonization and replication within wounds hinder the healing process. Common bacterial culprits isolated from these wounds are Staphylococcus aureus, Enterococcus faecalis, Pseudomonas aeruginosa, and others.

Flavonoids are often combined with other compounds that enhance cell growth for wound healing applications. A study demonstrated that quercetin increased keratinocyte proliferation. This study additionally encompassed clinical testing on 56 diabetic patients (equal numbers of men and women), where a gel-based nano formulation incorporating quercetin and oleic acid was administered to foot ulcers over eight months. Hyaluronic acid functioned as the comparative standard. The combined treatment showed significantly better results than hyaluronic acid alone, highlighting this flavonoid compound's potential as a beneficial flavonoid in wound care. The findings indicated that quercetin diminished cutaneous damage, enhanced dermal flexibility, and potentially contributes to the healing process of persistent wounds. The study proposed that regulating cytokines, growth factors, and proteases could support diabetic wound regeneration.

In this regard, Biochanin A (BCA) is an O-methylated isoflavone mainly found in alfalfa, cabbage, and red clover. BCA has various biological properties, including anti-inflammatory, antibacterial, and antioxidant effects.

BCA exhibits potent anti-inflammatory properties by inhibiting pro-inflammatory signaling and transcription factors. This involves reducing the activation of nuclear factor kappa B (NF-κB) and suppressing the levels of tumor necrosis factor-alpha (TNF-α), interleukin-1 beta (IL-1β), and interleukin-2 (IL-2). These actions contribute to the overall reduction of inflammation. BCA stimulates the nuclear factor E2-related factor2/antioxidant responsive element (Nrf2/ARE) pathway, leading to enhanced expression of antioxidant enzymes such as NAD(P)H quinone dehydrogenase 1 (NQO1) and heme-oxygenase 1 (HO-1). This activation helps reduce oxidative stress by increasing the activity of antioxidant markers such as superoxide dismutase (SOD) and catalase (CAT). Furthermore, BCA may influence the levels of malondialdehyde (MDA) and Nrf2, further contributing to its antioxidant effects.

In the view of the foregoing discussion, it is clearly portrayed that BCA can be utilizes as a potential therapeutic solution for promoting wound healing in case of diabetes, therefore the present invention provides a process for preparing Biochanin A (BCA)-loaded hydrogel for promoting wound healing in diabetic patients.

### SUMMARY OF THE INVENTION

The present disclosure relates to a process for preparing Biochanin A (BCA)-loaded hydrogel for promoting wound healing in diabetic patients. The present invention aims to investigate the therapeutic potential of Biochanin A (BCA), an isoflavone primarily found in red clover, in promoting wound healing in diabetic rats through modulation of antioxidative, inflammatory, and angiogenic pathways. BCA treatment enhanced the antioxidative defense system by increasing the expression of nuclear factor erythroid 2-related factor 2 (Nrf2) and its downstream targets including superoxide dismutase (SOD), catalase (CAT), and NAD(P)H: quinone oxidoreductase 1 (NQO1), while reducing malondialdehyde (MDA) levels. The inflammatory response was modulated through reduction of pro-inflammatory markers including interleukin-1β (IL-1β), interleukin-6 (IL-6), cyclooxygenase-2 (COX-2), tumor necrosis factor-alpha (TNF-α), and toll-like receptor 4 (TLR4). Notably, BCA enhanced wound healing by upregulating angiogenic factors including transforming growth factor-beta1 (TGF-β1), vascular endothelial growth factor-A (VEGF-A), platelet-derived growth factor receptor (PDGFR) and increasing collagen deposition collagen type I alpha 1 (Col 1A1) and collagen type IV alpha 1 (Col 4A1). These molecular changes corresponded with improved wound contraction and healing rates compared to untreated diabetic controls. Histological analysis confirmed increased granulation tissue formation, re-epithelialization, and neovascularization in the BCA-treated group. The obtained results demonstrated that BCA accelerates diabetic wound healing through its antioxidant and anti-inflammatory properties, suggesting its potential therapeutic value in treating diabetic wounds.

An object of the present invention is to provide a process for preparing Biochanin A (BCA)-loaded hydrogel for promoting wound healing in diabetic patients.

Another object of the present invention is to provide Biochanin A as a therapeutic agent for treating diabetic wounds through its antioxidant, anti-inflammatory, and pro-angiogenic properties, thereby reducing risks of lower limb amputation and associated economic and psychosocial burdens.

Another object of the present invention is to enhance the antioxidative defense system in diabetic wounds by increasing expression of nuclear factor erythroid 2-related factor 2 (Nrf2) and its downstream targets, including superoxide dismutase (SOD), catalase (CAT), and NAD(P)H: quinone oxidoreductase 1 (NQO1), while reducing malondialdehyde (MDA) levels.

Another object of the present invention is to modulate the inflammatory response in diabetic wounds by reducing pro-inflammatory markers such as interleukin-1β (IL-1β), interleukin-6 (IL-6), cyclooxygenase-2 (COX-2), tumor necrosis factor-alpha (TNF-α), and toll-like receptor 4 (TLR4).

Another object of the present invention is to promote angiogenesis and tissue repair in diabetic wounds by upregulating angiogenic factors including transforming growth factor-beta1 (TGF-β1), vascular endothelial growth factor-A (VEGF-A), and platelet-derived growth factor receptor (PDGFR).

Yet, another object of the present invention is to accelerate wound contraction, healing rates, granulation tissue formation, re-epithelialization, neovascularization, and collagen deposition of types I alpha 1 (Col 1A1) and IV alpha 1 (Col 4A1) in diabetic subjects.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a standard calibration curve of MDA, in accordance with an embodiment of the present disclosure;

FIG. 2 illustrates a schematic presentation of the principal of SOD assay, in accordance with an embodiment of the present disclosure;

FIG. 3 illustrates a table showing the RT master mix (per 20-μL reaction), in accordance with an embodiment of the present disclosure;

FIG. 4 illustrates a table showing the primers sequence, in accordance with an embodiment of the present disclosure;

FIG. 5A illustrates the wound healing process for different groups, in accordance with an embodiment of the present disclosure;

FIG. 5B illustrates the effects of vehicle-treated, BCA and marketed formulation on the wound contraction and % of wound contraction on day 14, in accordance with an embodiment of the present disclosure;

FIG. 6 illustrates the results of histopathological investigation using H&E staining and MTC staining, in accordance with an embodiment of the present disclosure;

FIG. 7 illustrates a table showing Histological features of wound healing in animals treated with vehicle-treated, BCA and marketed formulation on day 14, in accordance with an embodiment of the present disclosure;

FIG. 8A illustrates a graphical representation of effects of BCA on MDA concentration, in accordance with an embodiment of the present disclosure;

FIG. 8B illustrates a graphical representation of effects of BCA on SOD concentration, in accordance with an embodiment of the present disclosure;

FIG. 8C illustrates a graphical representation of effects of BCA on CAT concentration, in accordance with an embodiment of the present disclosure;

FIG. 9 illustrates Graphic presentation the effect of vehicle-treated, BCA, and marketed formulation on NQO1, and Nrf2 expression, in accordance with an embodiment of the present disclosure;

FIG. 10 illustrates Graphic presentation of effect of vehicle-treated, BCA and marketed formulation on inflammatory markers IL1β, IL6, COX-2, TNFα, and TLR4 levels in wound tissue, in accordance with an embodiment of the present disclosure;

FIG. 11 illustrates Graphic presentation of expression levels of angiogenic markers TGF-β, VEGF-A, and PDGFR in wound tissue, in accordance with an embodiment of the present disclosure;

FIG. 12A illustrates graphical representation of Assessment mRNA expression of Col 1A1, in accordance with an embodiment of the present disclosure;

FIG. 12B illustrates graphical representation of assessment of mRNA expression of Col 4A1, in accordance with an embodiment of the present disclosure; and

FIG. 13 illustrates a flow chart of a process for preparing a Biochanin-A (BCA) loaded hydrogel in accordance with an embodiment of the present disclosure.

Further, skilled artisans will appreciate that elements in the drawings are illustrated for simplicity and may not have been necessarily been drawn to scale. For example, the flow charts illustrate the process in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or process that comprises a list of steps does not include only those steps but may include other steps not expressly listed or inherent to such process or process. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, process, and examples provided herein are illustrative only and not intended to be limiting.

Embodiments of the present disclosure will be described below in detail with reference to the accompanying drawings.

The present invention relates to a process for preparing Biochanin A (BCA)-loaded hydrogel for promoting wound healing in diabetic patients, wherein the efficacy of the prepared hydrogel is evaluated by administrating the hydrogel in diabetes induced rats.

In the present invention following reagents are used: hydroxypropyl methyl cellulose (HPMC) for hydrogel preparation, diaminobenzidine: used for immunohistochemical staining, hematoxylin and Eosin (H and E) used for staining sections for histopathological examination. Masson's trichrome stain (MTC) used for staining sections for histopathological examination. hydrogen peroxide solution: used for immunohistochemical staining, methyl alcohol used for histopathological examination.

The present invention uses following primary antibodies: Multiclonal anti-IL-1 Beta (Cat #ab283818), Monoclonal anti-IL-6 (Cat #ab9324), Monoclonal anti-TNF-alpha (Cat #ab220210), Monoclonal anti-TGF beta1 antibody (Cat #ab215715), Monoclonal anti-VEGFA antibody (Cat #ab1316), Monoclonal anti-PDGFR alpha+PDGFR beta antibody (Cat #ab32570), Monoclonal anti-COX-2 (Cat #ab300668), Monoclonal anti-NQO1 (Cat #ab80588), Monoclonal anti-Nrf2 (Cat #ab313825), and Monoclonal anti-TLR4 (Cat #ab22048).

The present invention uses Kits for oxidative stress markers, colorimetric kits for assessment of malondialdehyde (MDA) content (Cat #700870), Catalase (CAT) content kit (Cat #707002) and superoxide dismutase (SOD) activity (Cat #706002). Mouse and Rabbit HRP/DAB (ABC) IHC detection kits (Cat #CTS005, and Cat #CTS002) are used for immunohistochemical assessment. RNA extraction kit (Cat #A27828, MagMAX mirVana and total RNA isolation kit) are used for real-time polymerase chain reaction (qPCR). cDNA Reverse Transcription Kit (Cat #4368814) are used for real-time polymerase chain reaction (qPCR). Taq PCR Master Mix kit (Cat #201445) are used for real-time polymerase chain reaction (qPCR).

For evaluating the efficacy of the prepared hydrogel for wound healing, winstar rats weighing between 190-230 g are used, wherein experimental animals were maintained under standard laboratory conditions characterized by humidity ranging from 30-70%, ambient temperature controlled at (22±2° C.), and alternating light-dark cycles of 12/12 hours. The ethical panel of research at the Faculty of Pharmacy, KAU, granted approval for the utilization of these animals in the research (Reference: PH-1445-27).

In an implementation, the present invention aims to utilize: an Accu-check Instant glucometer; Cooling Centrifuge; Freezer −80° C.; Microscope with camera; Plate reader; and Real-Time PCR System. The invention further aims to utilize, Image J, 1.46a software for histology and Immunohistochemistry, and GraphPad Prism version 1, for sketching graphs and performing statistical analyses.

For the preparation of Biochanin A loaded hydrogel, five g of Biochanin A (BCA) and 1.5 g of Hydroxypropyl methyl cellulose (HPMC) were weighed by analytical balance. Then, at room temperature, HPMC was dispersed in 100 ml of distilled water with stirring by a hotplate stirrer (LMS-1003), and the previously weighed BCA was gradually dissolved in the mixture with continuous stirring for 10 min. Beakers were then sealed with parafilm, stored in the refrigerator, and used after 2 days.

To prepare the vehicle gel at room temperature, 1.5 g of HPMC (5%) was dispersed in 100 ml of distilled water with stirring, then stored in the refrigerator, and used after 2 days.

Diabetes in rats was induced using a freshly prepared streptozotocin (STZ) solution in a cold citrate buffer with a concentration of 0.1 M and a pH of 4.5. The solution was injected intraperitoneally (I.P.) at a dose of 50 mg/kg. After 7 days, the levels of glucose in the blood were measured, only rats with glucose levels ranging from 200 to 300 mg/dl were allocated into the experimental groups.

After induction of diabetes and wounding, rats were sorted at random into 5 groups and labelled with the appropriate numbers (n=10 for each group). These were as follows:
Group 1: Normal control group, untreated non-diabetic rats.
Group 2: Negative control group, untreated diabetic rats.
Group 3: Rats were treated with the drug-free hydrogel (vehicle).
Group 4: Rats were treated only with BCA-loaded hydrogel.
Group 5: Positive control group, treated with MEBO® ointment, Gulf Pharmaceutical Industries (with-Sitosterol as the active ingredient, in a base of beeswax and sesame oil).

All treatments were applied topically for a total of 14 days. Wound diameters (WDs) were measured, and photographs of the wounds were taken on days 0, 3, 7, 10, and 14. On day 15, all the rats were sacrificed by decapitation, and then the skin around the wound was dissected. One section of each skin was preserved in 10% neutral formalin, while the other section was immediately frozen in liquid nitrogen and kept at −80° C. for the biochemical assessments.

To bring the diabetic rats in anesthetic state, ketamine and xylazine (100 mg/kg and 10 mg/kg body weight) was administered intraperitoneally. The shaved skin on the dorsal surface of their bodies was disinfected with ethanol (70%). After this, a 1×1 cm² area was removed from the dorsal surface of the rat, together with the epidermal, dermal, and subcutaneous layers of skin, as well as the connective tissue beneath the skin. Every diabetic rat was isolated in its own cage and provided with free access to water and food pellets.

The percentage of wound contraction was determined through the following formula: Wound contraction %=(WD on day 0−WD on day 14)/(WD on day 0)×100.

Immediately after sacrifice, skin tissues were excised out on ice, rinsed with a PBS solution, pH 7.4, to clean it from any red blood cells and clots, and then divided into two parts. One portion was kept in 10% neutral buffered formalin and the rest was placed at −80° C.

The Tissue is prepared for Histopathological and Immunohistochemical Analyses, wherein the wounded skin was dissected out and stored in 10% formalin for 24 h, after that skin tissue was cut into small pieces and embedded in hot paraffin wax blocks. A microtome was used to cut a thin segment that measured 4 micrometers in thickness. The tissues are further prepared for biochemical analysis, wherein following the meticulous rinsing of the collected skin specimens with cold saline, then carefully pressing the skin between two filter papers to dry the skin. The dried skins were then weighed and homogenized in phosphate-buffered saline (PBS) solution for obtaining 10% w/v tissue homogenate, followed by centrifugation of homogenate tissue at 3000 rpm for 20 minutes at 4° C. In addition to measuring the superoxide dismutase (SOD) and catalase (CAT) activities as antioxidant indicators, an assessment of malondialdehyde (MDA).

For histopathological examination, the skin tissues, kept in formalin, were washed in tap water then serial dilutions of alcohol (methyl, ethyl and absolute ethyl) and xylene were used for dehydration. Specimens were cleared in xylene and embedded in paraffin then formulated into paraffin blocks. After that, they were cut using a sledge tissue microtome into 4 μm thickness tissue sections. The tissue sections were then deparaffinized, rehydrated and stained with hematoxylin and eosin (H and E) and Masson's trichrome (MTC). The slides were examined by a pathologist, in a blind manner, with a Nikon light microscope to look for signs of possible structural damage. For the detection of the degree of tissue damage, healing, formation of new tissue, collagen deposition, and fibroblast proliferation each section was scored from − to +++, where −, +, ++, and +++ indicate none, weak, moderate, and high.

An oxidative stress marker assessment is carried out, including: Malondialdehyde (MDA) assessment; Superoxide Dismutase (SOD) Assay; and Catalase (CAT) Assay, which are described as under.

Lipid peroxidation was evaluated by examining the levels of TBARS that were articulated as MDA levels. MDA is the result of the course of action of lipid peroxidation and acts as an indicator of the process. The test principle depends on the colorimetric determination of a pink color, ensuing from the reaction of TBARS with thiobarbituric acid (TBA) in an acidic medium (pH 2-3) after boiling.

Kit content includes: Thiobarbituric acid (TBA); Acetic acid to prepare the color reagent; Sodium hydroxide to prepare the color reagent; MDA Standard for preparing the standard curve; and Sodium dodecyl sulfate (SDS).

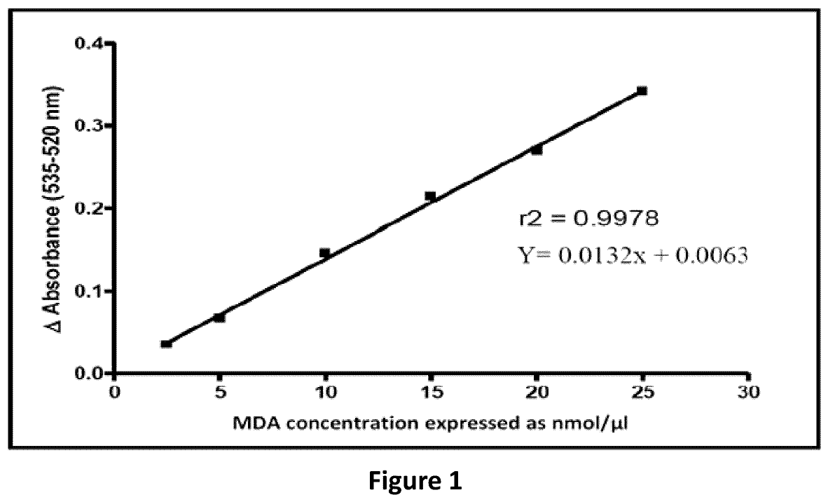

An amount of 100 μl of standard or sample was taken in to a 5 ml vial. An amount of 100 μl of TCA solution was added to all vials and mixed. An amount of 4 ml of TBA was added downside of each vial. Vials were boiled for an hour. To stop the reaction, vials were instantly detached and positioned in ice bath for 10 min. Vials were revolved for 10 min at 1,600×g at 4°. An amount of 150 μl from the supernatants was encumbered to a clear plate. Absorbance was examined at 530 nm. The values of MDA for each sample were calculated from the standard curve, as shown in FIG. 1.

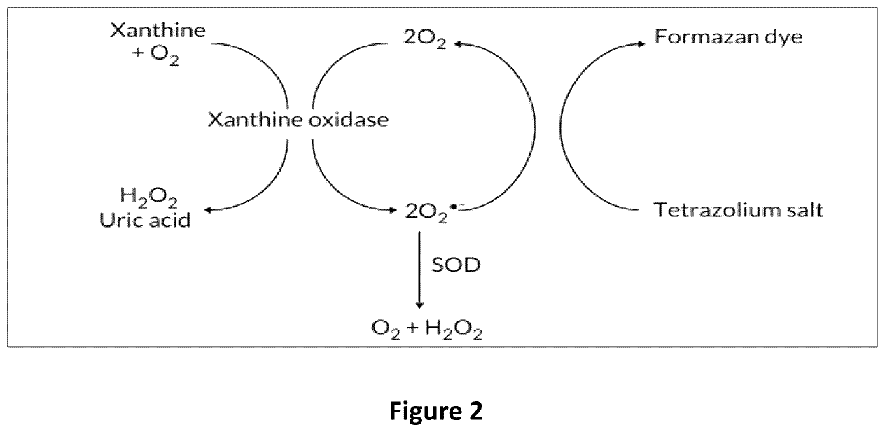

The test followed by utilizing tetrazolium salt to identify superoxide radicals released by hypoxanthine and xanthine oxidase. One SOD unit is described as the amount of enzyme dismutases 50% of superoxide radicals. The FIG. 2 shows the schematic presentation of the principal of SOD assay.

Kit Content includes: Assay Buffer; Sample Buffer; Radical Detector: tetrazolium salt solution; SOD Standard: bovine erythrocyte SOD (Cu/Zn); and Xanthine Oxidase.

Skin tissues were rinsed by PBS, pH 7.4, and removed red blood cells and clots. Tissues were homogenized in HEPES (4-(2-hydroxyethyl)-1 piperazine ethane sulfonic acid) buffer, pH 7.2 contains ethylene glycol tetra acetic acid, sucrose, and mannitol.

For performing assay, an aliquot of 200 μl of the radical detector and 10 μl of the standard were added in the designated wells. Then, 200 μl of the radical detector and 10 μl of the samples were added in the sample wells. By adding 20 μl of diluted xanthine oxidase to every well the reaction was initiated. The plate was covered and incubated on a shaker for 30 min. The absorbance was study at 450 nm.

The activities of SOD were determined using the following equation,

$$SOD ( U / ml ) = [ ( sample LR - y‐ intercept ) / slope \times ( 0.23 ml / 0.01 ml ) ] \times sample dilution$$

The antioxidant enzyme catalase exhibits ubiquitous distribution across most aerobic cells. This enzyme, abbreviated as CAT, functions primarily in neutralizing hydrogen peroxide (H₂O₂), which belongs to the reactive oxygen species (ROS) category and originates as a toxic byproduct from both regular aerobic metabolic processes and pathological ROS generation. The enzymatic action of catalase facilitates the transformation of a pair of H₂O₂ molecules into molecular oxygen and two water molecules, demonstrating its catalytic functionality. Additionally, CAT exhibits peroxidatic activity whereby it can utilize low molecular weight alcohols as electron donors. A distinctive characteristic of CAT is its specificity for aliphatic alcohols as substrates, a property not shared by other enzymes possessing peroxidatic activity, which cannot utilize these particular substrates.

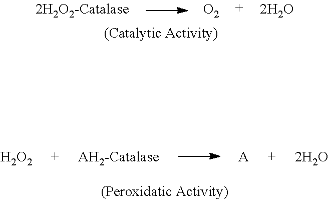

In humans, the highest levels of CAT are found in liver, kidney, and erythrocytes, where it is believed to account for the majority of H₂O₂ decomposition.

Kit Content includes: CAT Assay Buffer; CAT Sample Buffer; CAT Formaldehyde Standard; CAT Control; CAT Potassium Hydroxide; CAT Hydrogen Peroxide; CAT Purpald (Chromogen); and CAT Potassium Periodate.

For the formaldehyde standard wells preparation, a combination of Catalase Assay Buffer (1X) (100 μl), methanol (30 μl), and standard solution (20 μl) was introduced into the designated plate wells. The positive control wells were established by combining Catalase Assay Buffer (1X) (100 μl), methanol (30 μl), and diluted Catalase (Control) (20 μl) in duplicate wells. For sample wells, Catalase Assay Buffer (1X) (100 μl), methanol (30 μl), and sample material (20 μl) were introduced into duplicate wells. When required, sample concentration adjustments were performed either through dilution using Catalase Sample Buffer (1X) or concentration via an Amicon centrifuge concentrator featuring a 100,000 molecular weight cut-off threshold.

To initiate the enzymatic reactions, diluted Hydrogen Peroxide (20 μl) was expeditiously introduced to all experimental wells, with precise documentation of the initiation time. Subsequently, the plate was sealed with a cover and subjected to room temperature incubation with agitation for 20 minutes. Following this period, Potassium Hydroxide (30 μl) was applied to each well to halt the reaction, immediately followed by the addition of Catalase Purpald (Chromogen) (30 μl) to each well. The covered plate then underwent a 10-minute room temperature incubation with continuous agitation. In the final preparatory step, Catalase Potassium Periodate (10 μl) was introduced to each well, followed by an additional five-minute room temperature incubation with agitation. The spectrophotometric analysis was subsequently conducted by measuring absorbance at 540 nm.

The CAT activity is calculated using the formula given under:

$$CAT Activity = ( μM of Sample / 20 min . ) \times Sample dilution = \text{nmol/min/ml}$$

Immunohistochemical analyses are performed, wherein the protein expressions of IL-1β, IL-6, anti-TNF, TGF-1β, VEGFA, PDGER-alpha+beta, COX-2, NQO1, Nrf2, and TLR4 were assessed using the immunohistochemical protein assessment technique.

Immunohistochemical protein detection and localization depends on the reaction between the target protein (Antigen) and specific antibody. Enzymes, such as Horseradish Peroxidase (HRP) is commonly used to catalyze a color-producing reaction. And the color can be detected by photographing using light microscope then quantified using image analysis software. Deparaffinized tissue sections were rehydrated with different ethanol concentration and boiled in citrate buffer (pH 6.0) for 10 minutes. After deparaffinization, sections were treated with 3% H₂O₂ in distilled H₂O for 10 min to quench the endogenous peroxide. A concentration of 10% goat serum was used to block nonspecific binding sites. The sections were then incubated for two hours in TBS containing 5% BSA, and then exposed overnight at 4° C. to the primary antibodies: Polyclonal anti-IL-6, Monoclonal anti-TNF, Monoclonal anti-TGF beta1 antibody, Monoclonal anti-VEGFA antibody and Monoclonal anti-PDGFR-beta, This was followed by incubation with biotinylated goat-anti-rabbit IgG, secondary antibodies, for 10 min. Sections were washed with PBS 3 times of 3 min each. This was followed by incubation for 10 min with streptavidin HRP. Antibody binding sites were detected by incubation with diaminobenzidine—H₂O₂ solution. Image analysis to calculate the optical density (OD) and area percent (A %) was performed using ImageJ software.

For the assessment of mRNA expression of Col1A1 and Col4A1, qPCR is performed. PCR is based on DNA polymerase enzyme-exponential copying of a part of a DNA molecule specifically increasing a target. Real time PCR detection process depend on changes in fluorescence, which are proportional to the increase of the target. Wounded skin sections were homogenized by utilizing an ultra-probe sonicator. RNA extraction was performed using triazole solution, and an extraction kit of RNA.

Kit components includes: Proteinase K, 50 mg/mL; Lysis/Binding Enhancer; TURBO DNase; Lysis Buffer; PK Digestion Buffer; RNA Binding Beads; Wash Solutions; Rebinding Buffer; DNase Buffer; Elution Buffer; Processing Plate; and Elution Plates.

Homogenized samples were mixed with magnetic beads. The beads were washed with wash buffers. RNA was then eluted off the beads. Then, a reverse transcription kit (Applied Biosystems, Foster City, CA, USA) was utilized for the synthesis of cDNA.

Reagents include: RT Buffer; RT Random Primers; dNTP Mix (100 mM); MultiScribe™ Reverse Transcriptase, 50 U/μL; and RNase Inhibitor.

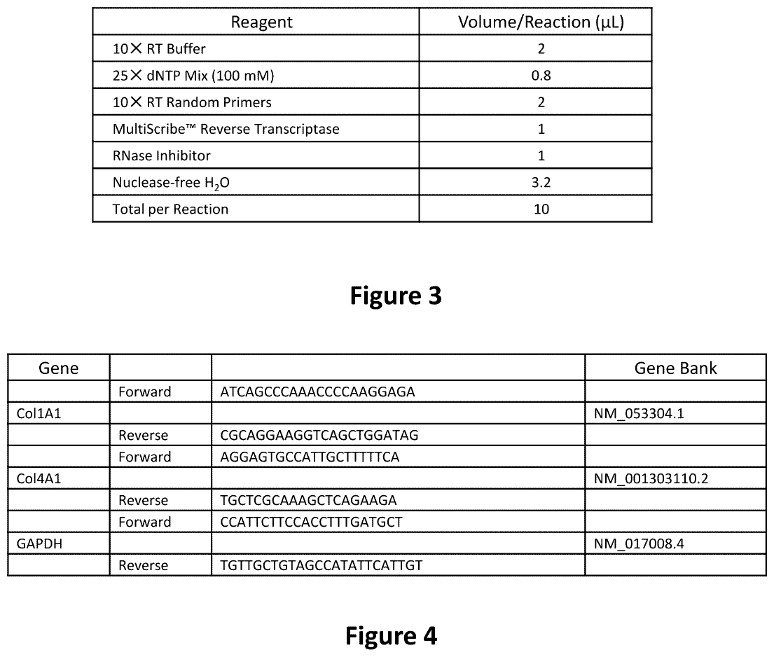

The RT master mix (per 20-μL reaction) is prepared as shown in the table shown in FIG. 3. Ten microliters of 2× RT master mixture were pipetted into each individual tube. Ten microliters of RNA sample were pipetted into each well, pipetting up and down two times to mix. The tubes were sealed and briefly centrifuged to spin down the contents and eliminate any air bubbles. The thermal cycler conditions were programmed as following: at step 1, temperature is 25° C. and time is 10 min, at step 2, temperature is 37° C. and time is 120 min, at step 3, temperature is 85° C. and time is 5 min, and at step 4, temperature is 4° C., and time is kept hold. Reactions were loaded into the thermal cycler and the reverse transcription run was started. For Amplification the following kit components were used: Taq DNA Polymerase; QIAGEN PCR Buffer; MgCl₂; and Ultrapure dNTPs. For each experimental sample, a mixture for real-time PCR analysis was formulated. Following preparation, the reaction plate underwent sealing, gentle agitation to ensure proper mixing, and brief centrifugation to concentrate the contents and remove any entrapped air bubbles. After configuring the appropriate thermal cycling parameters, the prepared plate was processed using an Applied Biosystems Step One real-time quantitative PCR system. Upon completion of the amplification protocol, individual melting curves were generated for each primer set utilized. The quantitative data generated from the RT-PCR procedure underwent subsequent analysis through the proprietary software integrated with the Applied Biosystems real-time PCR instrumentation.

The primer sequence of gene, Col1A1, Col4A1, and GAPDH are shown in table shown in FIG. 4.

Gene expression changes were calculated by the comparative cycle threshold (Ct) process and the values were normalized to endogenous reference GAPDH according to the following equations.

$$Δ {Ct}_{sample} = {Ct}_{gene} of interest - {Ct}_{housekeeping gene}$$

$$ΔΔ Ct = Δ {Ct}_{sample} - Δ {Ct}_{control} group$$

$$RQ = 2 - ΔΔ Ct$$

The experimental results are expressed as means±standard deviation (SD). Statistical evaluation between experimental groups was conducted through Analysis of Variance (ANOVA), with subsequent application of Tukey's post hoc test for multiple comparisons. The statistical computations and analyses were executed using GraphPad Prism software, version 10. Statistical significance was established at P-values 0.05> for all analytical comparisons.

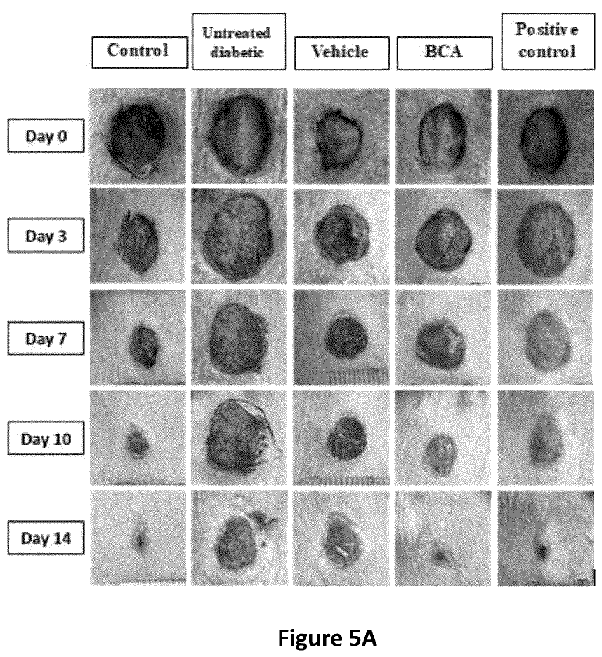

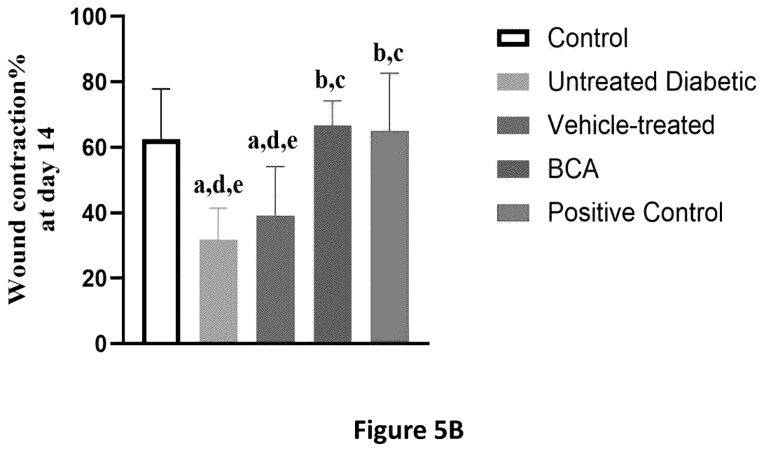

As shown in FIG. 5A, all experimental groups, including control, untreated diabetic, vehicle-treated, BCA-treated, and positive control groups, exhibited comparable initial wound areas on day 0. Throughout the observation period (days 3, 7, 10, and 14), the topical application of BCA demonstrated progressive wound contraction and healing compared to other groups. The wound healing process was monitored through macroscopic evaluation and quantification of wound contraction percentage.

As shown in FIG. 5B, by day 14, the BCA-treated group showed significant improvement in wound contraction (approximately 66.7%) compared to the untreated diabetic group (approximately 31.7%). This represents a 2.1-fold increase in wound contraction rate with BCA treatment. The control group exhibited about 62.5% wound contraction, while the positive control group showed comparable results to the BCA-treated group (approximately 65%). The vehicle-treated group, demonstrated moderate improvement (approximately 39.2%) compared to the untreated diabetic group, but remained less effective than BCA treatment.

The enhanced wound healing effect observed in the BCA-treated group was evidenced by the progressive reduction in wound size and improved tissue regeneration throughout the study period. The macroscopic images demonstrate that BCA treatment accelerated the wound closure process, with visible improvements in wound appearance and size reduction starting from day 3 and continuing through day 14.

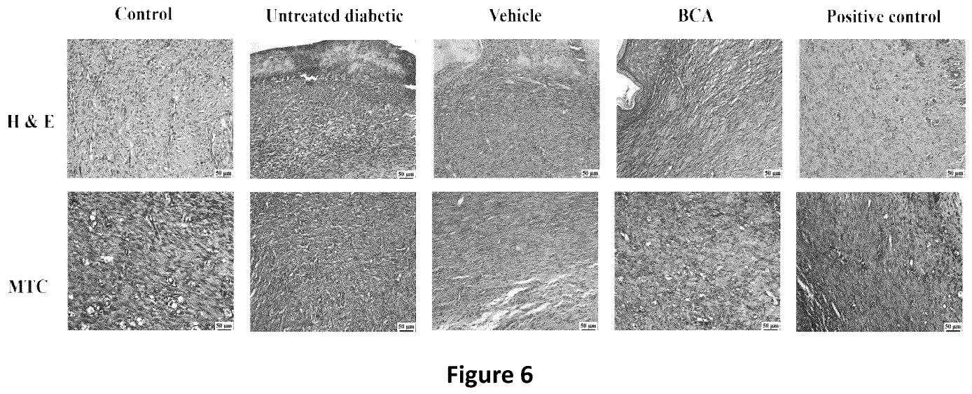

As shown in FIG. 6, effective wound healing was observed in the control group, characterized by abundant collagen-rich, well-vascularized granulation tissue filling the wound gap. In the untreated diabetic group, an intense inflammatory reaction was noted at the wound area, with haphazardly arranged granulation tissue deficient in collagen fibers. The wound surface was covered by necrotic tissue debris and inflammatory cell infiltration. Re-epithelialization was absent in all examined sections, with aggregations of bacterial colonies. Similar results were detected in the vehicle-treated group. The positive control (Mebo) group revealed an enhanced wound healing process, exhibiting improved wound closure with numerous blood vessels beneath the newly formed epidermal layer. Similarly, the BCA group showed enhanced re-epithelialization; optimal wound remodeling and contraction were observed, and the wound area was filled with well-vascularized, collagen-rich fibrovascular tissue with only a mild inflammatory reaction. Staining of skin sections using Masson's trichrome stain (MTC) was used to assess collagen deposition as a marker of wound healing. A high degree of collagen deposition and organization within the wound gap indicated an accelerated rate of wound healing. Marked elevation of collagen fibers was observed in the BCA-treated group, followed by the Mebo-treated group, when compared to diabetic controls.

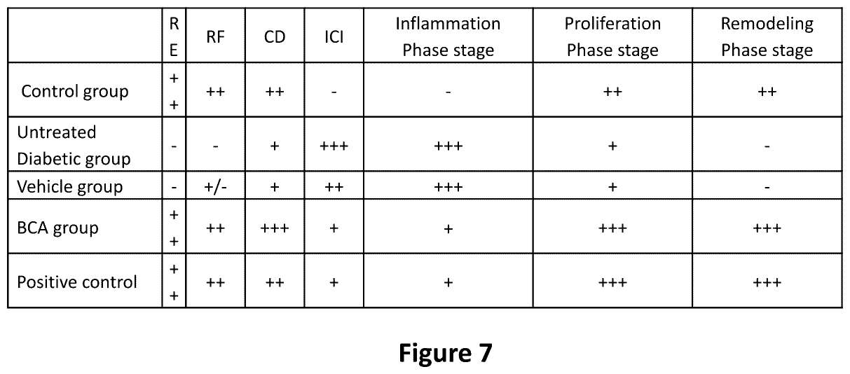

The Histological features of wound healing in animals treated with vehicle-treated, BCA and marketed formulation on day 14, are shown in table shown in FIG. 7, wherein RE=reepithelization, PF=proliferation of fibroblast, CD=collagen deposition, ICI=inflammatory cell infiltration, −, +, ++, and +++ indicate none, weak, moderate, and high.

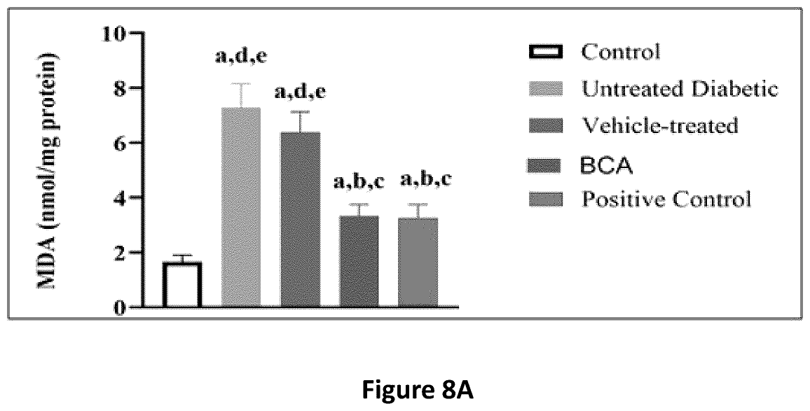

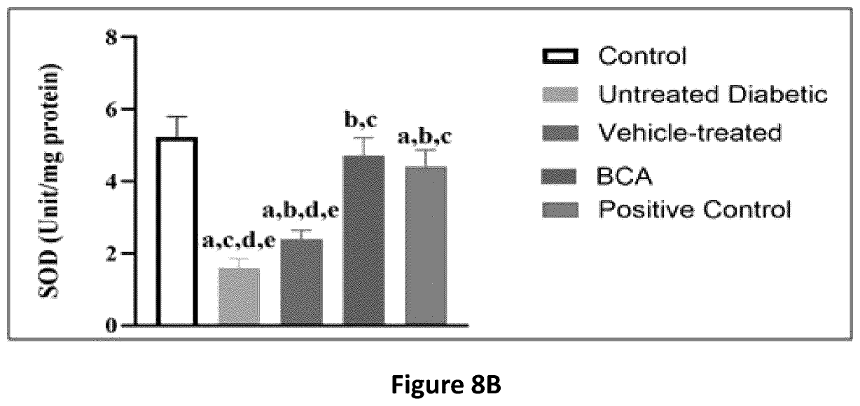

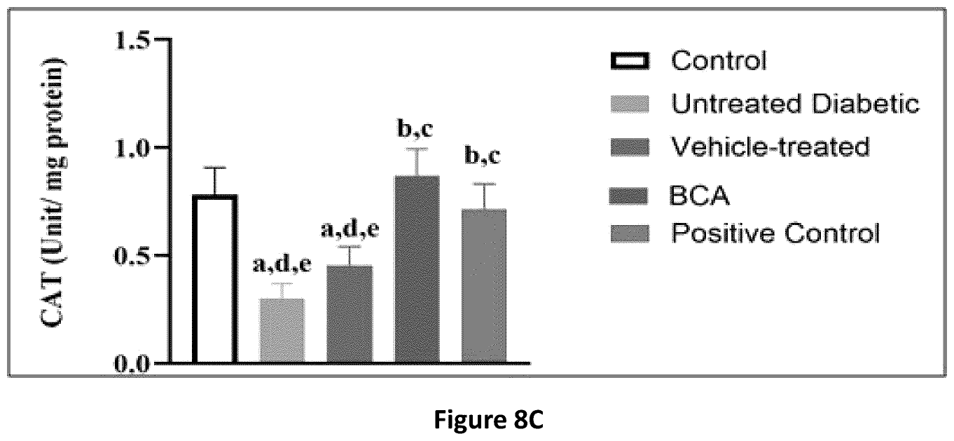

The provided results in FIG. 8A describe MDA levels in different experimental groups. MDA levels were markedly elevated in the untreated diabetic group (approximately 4.5-fold increase) compared to the control group. Treatment with BCA significantly reduced MDA levels by about 55% compared to the untreated diabetic group, demonstrating levels similar to those observed in the positive control group. The vehicle-treated group showed minimal improvement in MDA levels compared to the untreated diabetic group.

As shown in FIG. 8B, SOD activity was notably reduced in the untreated diabetic group by about 69.5% compared to the control group. BCA treatment significantly enhanced SOD activity, showing restoration to approximately 84% of control levels. The BCA-treated group exhibited 2.76-fold higher SOD activity compared to the untreated diabetic group. The vehicle-treated group showed minimal improvement in SOD activity compared to the untreated diabetic group.

As presented in FIG. 8C, the CAT activity was significantly decreased in the untreated diabetic group by approximately 62% compared to the control group. Treatment with BCA showed a remarkable restoration of CAT activity, reaching approximately 91% of the control group levels. The BCA-treated group demonstrated 2.4-fold higher CAT activity compared to the untreated diabetic group. The vehicle-treated group showed minimal improvement in CAT activity compared to the untreated diabetic group.

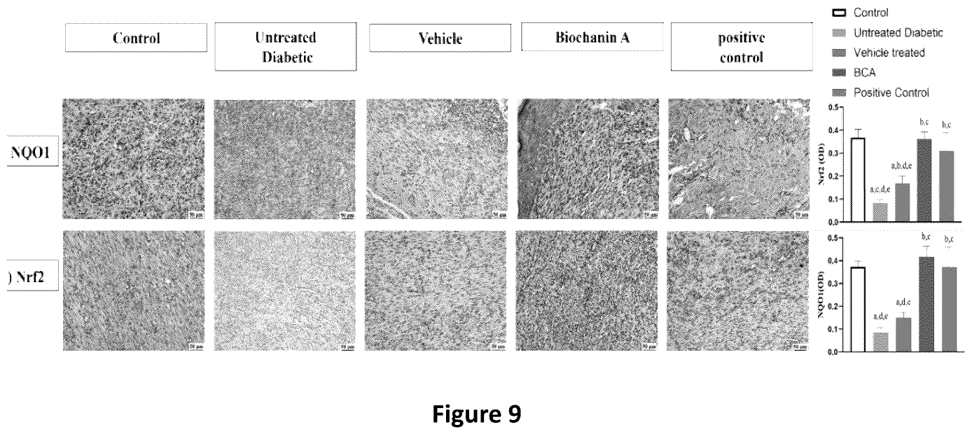

The results presented in FIG. 9 illustrate the NQO1 protein expression levels in the wound tissues of the different experimental groups. A marked decrease in NQO1 levels was observed in the untreated diabetic group, being approximately 4.4-fold lower compared to the control group. Treatment with BCA led to a substantial enhancement in NQO1 expression, with an approximately 4.9-fold increase compared to the untreated diabetic group. This BCA treatment restored NQO1 levels to values comparable to those observed in the control group. Similarly, the positive control group (Mebo) showed a significant increase in NQO1 levels, approximately 4.4-fold higher than the untreated diabetic group, reaching levels comparable to the control group. The vehicle-treated group showed a slight improvement in NQO1 levels (approximately 1.8-fold increase compared to untreated); however, these levels remained significantly lower compared to the BCA-treated group.

FIG. 9 illustrates the protein expression levels of Nrf2 in the wound tissues of the different experimental groups. A marked decrease in Nrf2 levels was observed in the untreated diabetic group, being approximately 4.4-fold lower compared to the control group. Treatment with BCA led to a significant enhancement in Nrf2 expression, with an increase of approximately 4.3-fold compared to the untreated diabetic group. This BCA treatment restored Nrf2 levels to values comparable to those observed in the control group. The positive control group (Mebo) also showed a notable improvement in Nrf2 levels, with an increase of approximately 3.7-fold compared to the untreated diabetic group. The vehicle-treated group exhibited a slight improvement in Nrf2 levels; however, these levels remained lower compared to the BCA-treated group.

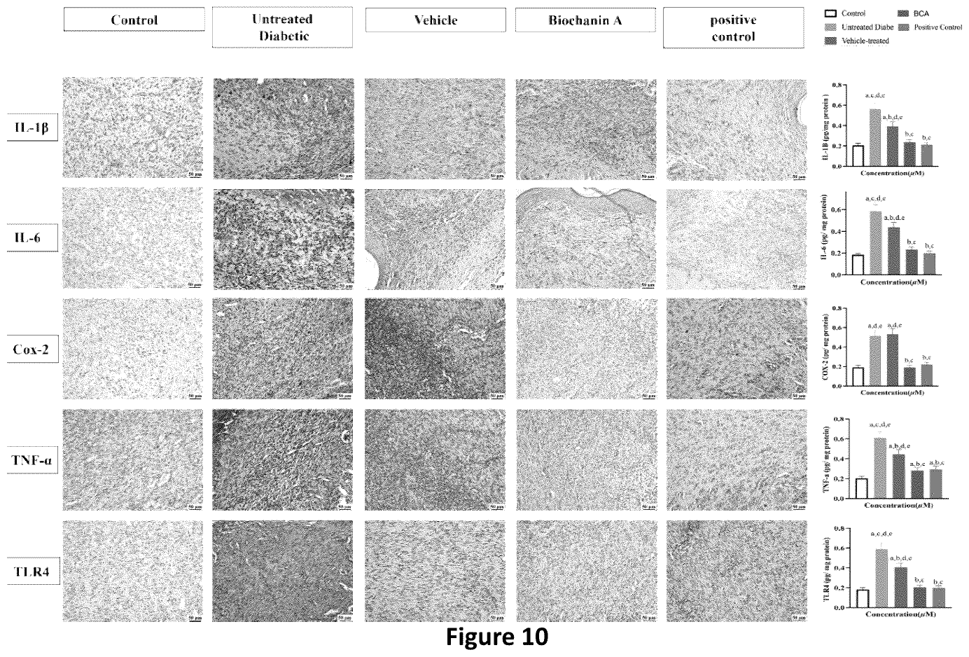

As shown in FIG. 10, the impact of BCA treatment on IL-1β levels in diabetic skin tissue. The data indicate that the untreated diabetic group showed a significant increase in IL-1β concentration (2.7-fold higher) compared to the control group. Notably, pre-treatment with BCA significantly prevented the rise in IL-1β concentration by approximately 57.9% compared to the untreated diabetic group. The vehicle-treated group showed a moderate reduction of 30% compared to the untreated diabetic group. When compared to the vehicle-treated group, BCA treatment demonstrated better efficacy with a significant 39.8% reduction in IL-1β levels. The positive control group showed similar effectiveness to BCA treatment, reducing IL-1β levels by 62.5% compared to the untreated diabetic group.

The provided results in FIG. 10 also describe the impact of BCA treatment on IL-6 levels in diabetic skin tissue. The data indicate that the untreated diabetic group showed a significant increase in IL-6 concentration (3.2-fold higher) compared to the control group. Notably, pre-treatment with BCA significantly prevented the rise in IL-6 concentration by approximately 60.2% compared to the untreated diabetic group. The vehicle-treated group showed only a modest reduction of 25.4% compared to the untreated diabetic group. When compared to the vehicle-treated group, BCA treatment demonstrated superior efficacy with a significant 46.7% reduction in IL-6 levels. The positive control group showed similar effectiveness to BCA treatment, reducing IL-6 levels by 66.2% compared to the untreated diabetic group.

The FIG. 10 further highlights the effects of BCA treatment on COX-2 levels in diabetic skin tissue. The results reveal that the untreated diabetic group exhibited a significant increase in COX-2 concentration (2.68-fold higher) compared to the control group. Interestingly, pre-treatment with BCA effectively reduced COX-2 levels by 63.1% compared to the untreated diabetic group. The vehicle-treated group demonstrated a slight reduction of 3.1% compared to the untreated diabetic group, indicating limited efficacy. In comparison to the vehicle-treated group, BCA treatment showed a remarkable improvement, reducing COX-2 levels by 64.3%. The positive control group also showed significant efficacy, reducing COX-2 levels by 57.3% compared to the untreated diabetic group.

As further presented in FIG. 10, the data show the impact of BCA treatment on TNF-α levels in diabetic skin tissue. The data indicate that the untreated diabetic group showed a significant increase in TNF-α concentration (3-fold higher) compared to the control group. Notably, pre-treatment with BCA significantly prevented the rise in TNF-α concentration by approximately 54.1% compared to the untreated diabetic group. The vehicle-treated group showed a moderate reduction of 27.1% compared to the untreated diabetic group. When compared to the vehicle-treated group, BCA treatment demonstrated superior efficacy with a significant 37.1% reduction in TNF-α levels. The positive control group showed similar effectiveness to BCA treatment, reducing TNF-α levels by 52% compared to the untreated diabetic group.

The experimental findings presented in FIG. 10 further demonstrate the effects of BCA on TLR4 expression in diabetic skin tissue. The data show that the untreated diabetic group exhibited a marked elevation in TLR4 levels (3.23-fold higher) compared to the control group. Notably, BCA pre-treatment resulted in a substantial reduction of TLR4 levels by 65.5% compared to the untreated diabetic group. The vehicle-treated group showed a moderate reduction of 31.5% compared to the untreated diabetic group. When compared to the vehicle-treated group, BCA treatment demonstrated superior efficacy with a significant 49.6% reduction in TLR4 levels. The positive control group showed similar effectiveness to BCA treatment, reducing TLR4 levels by 66.5% compared to the untreated diabetic group.

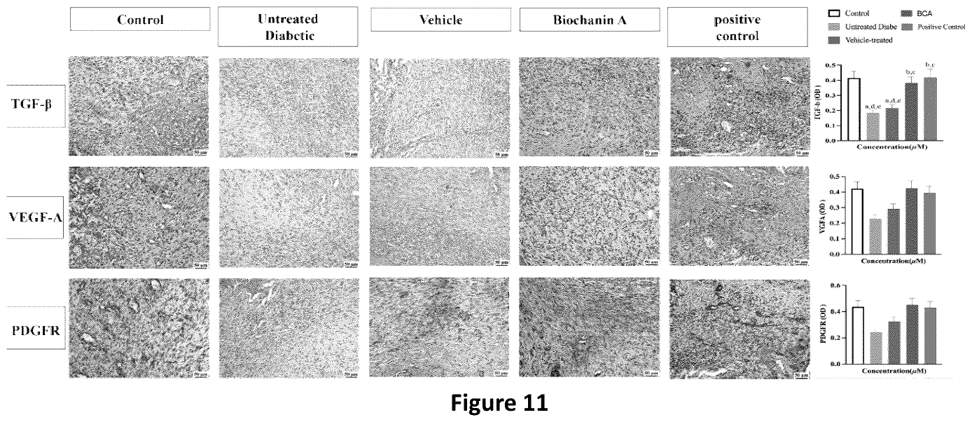

In the present invention, the untreated diabetic group and vehicle-treated group showed a significant reduction in TGF-β1 levels compared to the control group. Treatment with BCA significantly elevated TGF-β1 expression by 2.09-fold compared to the untreated diabetic group and 1.77-fold compared to the vehicle-treated group. The positive control group demonstrated comparable efficacy to BCA, showing a 2.29-fold increase in TGF-β1 levels compared to the untreated group, as shown in FIG. 11.

As also illustrated in FIG. 11, the untreated diabetic group and vehicle-treated group exhibited a marked decrease in VGFA levels relative to the control group. BCA treatment significantly enhanced VGFA expression by 1.85-fold compared to the untreated diabetic group and 1.45-fold compared to the vehicle-treated group. The positive control group demonstrated a comparable efficacy, with a 1.72-fold increase in VGFA levels relative to the untreated group.

As further shown in FIG. 11, the untreated group and vehicle group exhibited significant reductions in PDGFR levels compared to the control group. BCA treatment induced a marked 1.86-fold increase in protein expression relative to the untreated group and 1.39-fold versus the vehicle group, achieving results comparable to the positive control group (1.77-fold).

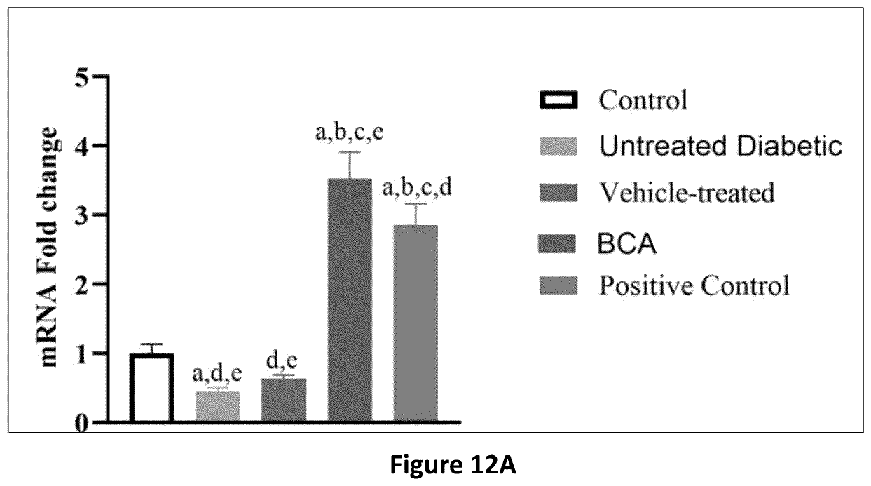

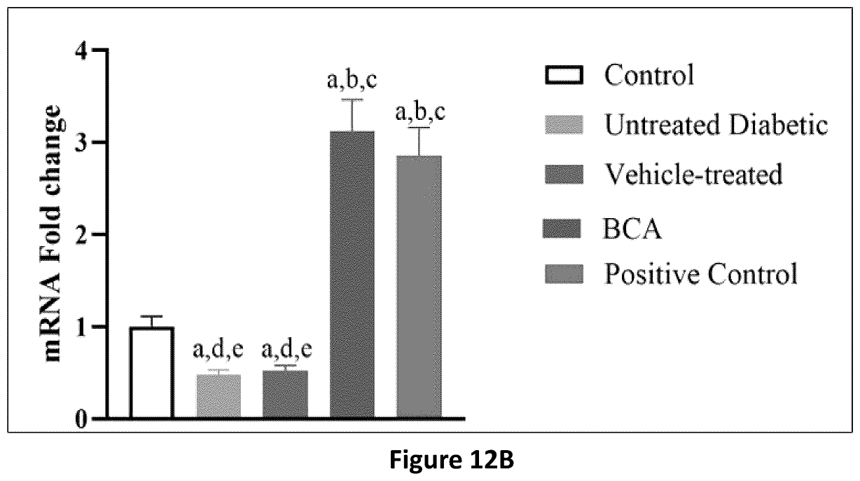

The analysis of Col 1A1 gene expression revealed significant variations across different experimental groups. The control group exhibited baseline expression levels 1-fold change, while the untreated diabetic group showed markedly reduced expression. Vehicle-treated samples demonstrated similarly low expression levels comparable to the untreated diabetic group. Notably, BCA treatment resulted in a substantial increase in Col 1A1 expression, reaching approximately 3.1-fold change, which was even slightly higher than the positive control group's 2.8-fold change, as shown in FIG. 12A.

In the present study, the analysis of Col 4A1 gene expression demonstrated significant variations across different experimental groups. The control group exhibited baseline expression levels 1-fold change, while the untreated diabetic group showed markedly reduced expression, 0.4-fold change. Vehicle-treated samples demonstrated minimal improvement 0.6-fold change compared to the untreated diabetic group. BCA treatment resulted in a substantial increase in Col 4A1 expression, reaching approximately 3.5-fold change, which was significantly higher than the positive control group, 2.8-fold change, as shown in FIG. 12B.

The wound healing features of BCA were evaluated in streptozotocin-induced diabetic rats, as shown in FIG. 5A and FIG. 5B, wherein the results indicated that BCA offered a significantly expedited wound healing activity. This was confirmed by histological examinations, which indicated that the BCA wounds showed almost complete healing as they reached the final phases of healing with re-modelling and collagen deposition (FIG. 6 and table shown in FIG. 7).

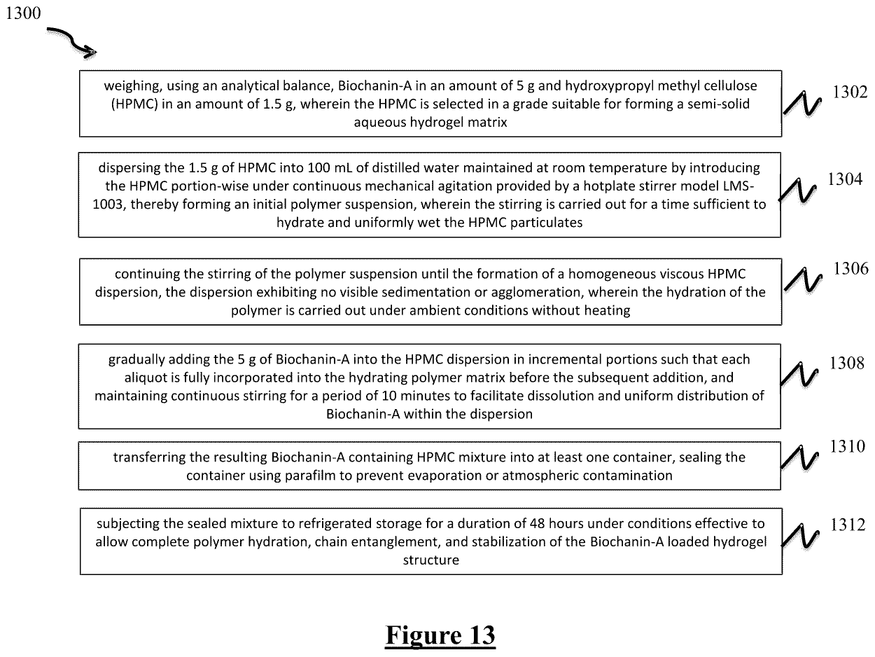

Referring to FIG. 13, a flow chart for a process for preparing a Biochanin-A (BCA) loaded hydrogel, the process comprising the steps of is illustrated. The process 1300 comprises:
At step 1302, the process 1300 includes weighing, using an analytical balance, Biochanin-A in an amount of 5 g and hydroxypropyl methyl cellulose (HPMC) in an amount of 1.5 g, wherein the HPMC is selected in a grade suitable for forming a semi-solid aqueous hydrogel matrix;
At step 1304, the process 1300 includes dispersing the 1.5 g of HPMC into 100 mL of distilled water maintained at room temperature by introducing the HPMC portion-wise under continuous mechanical agitation provided by a hotplate stirrer model LMS-1003, thereby forming an initial polymer suspension, wherein the stirring is carried out for a time sufficient to hydrate and uniformly wet the HPMC particulates;
At step 1306, the process 1300 includes continuing the stirring of the polymer suspension until the formation of a homogeneous viscous HPMC dispersion, the dispersion exhibiting no visible sedimentation or agglomeration, wherein the hydration of the polymer is carried out under ambient conditions without heating;
At step 1308, the process 1300 includes gradually adding the 5 g of Biochanin-A into the HPMC dispersion in incremental portions such that each aliquot is fully incorporated into the hydrating polymer matrix before the subsequent addition, and maintaining continuous stirring for a period of 10 minutes to facilitate dissolution and uniform distribution of Biochanin-A within the dispersion;
At step 1310, the process 1300 includes transferring the resulting Biochanin-A containing HPMC mixture into at least one container, sealing the container using parafilm to prevent evaporation or atmospheric contamination; and
At step 1312, the process 1300 includes subjecting the sealed mixture to refrigerated storage for a duration of 48 hours under conditions effective to allow complete polymer hydration, chain entanglement, and stabilization of the Biochanin-A loaded hydrogel structure,
wherein the distilled water used for dispersing HPMC has a conductivity below 10 μS/cm and is added in a single 100 mL volume prior to introducing the polymer, and wherein the dispersion comprises maintaining the water at 22-25° C. such that no thermal gelation of HPMC occurs, and wherein the dispersing of the HPMC comprises introducing the HPMC powder slowly into the central vortex of the mechanical stirrer to avoid clumping, and wherein the agitation speed is maintained sufficiently high to prevent the polymer from forming surface aggregates or undispersed globules, and wherein the Biochanin-A is added gradually into the hydrated HPMC matrix in incremental masses not exceeding 0.5 g at a time to enable dissolution within the viscous dispersion, and wherein the stirring is performed in a manner preventing sedimentation of Biochanin-A particulates within the hydrogel precursor.

In an embodiment, the continuous stirring for dissolution of Biochanin-A is performed for at least 10 minutes without interruption, and wherein the resulting mixture achieves uniform macroscale distribution of Biochanin-A within the HPMC network prior to sealing, wherein sealing the hydrogel precursor with parafilm comprises covering the open surface of the container fully to prevent moisture exchange, oxidative exposure, and microbial ingress, thereby preserving the physicochemical stability of the hydrogel during the 48-hour hydration period, and wherein the refrigeration is carried out at a temperature between 2° C. and 8° C. for a minimum of 48 hours, such that full polymer hydration, viscosity stabilization, and network formation occur as the HPMC chains interact with aqueous media under low-temperature equilibration.

In this embodiment, the system ensures complete incorporation of Biochanin-A into the hydrogel matrix through uninterrupted continuous stirring for a minimum duration of ten minutes. Maintaining continuous agitation is technically significant because Biochanin-A, being a poorly water-soluble flavonoid, requires sustained mechanical shear to disperse evenly throughout the progressively hydrating HPMC network. If stirring is discontinued prematurely, the hydrophobic particles tend to rise, sediment, or form clusters, resulting in visible heterogeneity and non-uniform drug loading. By contrast, uninterrupted stirring maintains a consistent vortex that draws Biochanin-A into the internal fluid layers of the forming polymer dispersion, thereby facilitating uniform macroscale distribution within the entire hydrogel body. Experimental observations show that when stirring is maintained for at least ten minutes, the resulting dispersion exhibits no color banding, particulate accumulation, or concentration gradients, confirming homogeneous incorporation.

Following homogenization, the hydrogel precursor is transferred to a container and sealed completely with parafilm. The sealing step is not merely a procedural measure but a functional requirement that directly affects the hydrogel's stability during the 48-hour hydration phase. Parafilm, when stretched tightly over the container opening, forms a conformal barrier that prevents moisture exchange with the environment, preventing dehydration of the hydrogel surface and avoiding changes in polymer concentration that would otherwise alter viscosity. The barrier also shields Biochanin-A from oxidative exposure, which is critical because flavonoids exhibit sensitivity to ambient oxygen and can undergo structural degradation if the precursor mixture is left unsealed. Additionally, the parafilm covering acts as a microbial ingress barrier, reducing the risk of contamination during the long equilibration period. As the hydrogel will remain undisturbed during refrigeration, maintaining aseptic integrity is essential to ensure reproducibility and safety for downstream applications.

The sealed container is then subjected to refrigeration at a temperature maintained between 2° C. and 8° C. for no less than 48 hours. This specific temperature range is chosen because HPMC undergoes hydration, swelling, and viscosity development at a controlled rate under low-temperature conditions. At elevated temperatures, premature gelling or local thickening may occur before complete polymer chain expansion, resulting in structural inconsistencies throughout the matrix. Under refrigeration, however, hydration progresses gradually, allowing water molecules to diffuse uniformly into the polymer chains, enabling full entanglement and stable gel network formation. The 48-hour period ensures that the polymer chains complete their swelling cycle, reach viscosity equilibrium, and align to form a cohesive three-dimensional matrix in which Biochanin-A remains entrapped uniformly. This prolonged equilibration period also stabilizes the drug-polymer interactions and prevents post-formulation phase separation.

Together, the uninterrupted ten-minute stirring, the parafilm sealing, and the controlled low-temperature hydration synergistically ensure that the resulting hydrogel demonstrates consistent viscosity, structural integrity, chemical stability of Biochanin-A, and reproducible drug distribution across batches. This embodiment therefore provides both the procedural and physicochemical conditions necessary to enable a technically sound and industrially applicable hydrogel preparation process.

In an embodiment, further comprising preparing a drug-free vehicle hydrogel by dispersing 1.5 g of HPMC in 100 mL of distilled water under identical stirring conditions, sealing the mixture, and refrigerating the hydrogel for 48 hours, wherein no Biochanin-A is added to said vehicle hydrogel, wherein the hydrogel precursor is stored in chemically inert laboratory glass beakers sealed with parafilm to provide an airtight enclosure, and wherein the beakers are placed in refrigeration without agitation to allow undisturbed polymer settling and full hydration, and wherein the stirring of HPMC and Biochanin-A is conducted using the LMS-1003 hotplate stirrer without activating the heating module, and wherein the stirrer maintains uniform rotational motion throughout the dispersion and dissolution stages.

In this embodiment, the process additionally includes the preparation of a drug-free vehicle hydrogel that serves as a comparative control to evaluate the rheological, structural, and functional properties of the Biochanin-A loaded hydrogel. The drug-free hydrogel is formulated by dispersing 1.5 g of HPMC in 100 mL of distilled water under the same mechanical stirring conditions used for the drug-loaded formulation. Maintaining identical dispersion parameters ensures that any differences observed in viscosity, transparency, swelling behavior, or mechanical stability arise solely from the incorporation of Biochanin-A, thereby providing a scientifically valid reference for assessing the synergistic effect of drug-polymer interactions. The LMS-1003 hotplate stirrer is used without activating the heating module, ensuring that the hydration of HPMC occurs strictly through mechanical agitation at ambient temperature. This is essential because HPMC exhibits thermogelation properties; heating during dispersion would alter polymer chain expansion kinetics and compromise consistency between the vehicle and drug-loaded hydrogels.

During its preparation, the vehicle hydrogel precursor is transferred into chemically inert borosilicate glass beakers, whose non-reactive surfaces ensure that no polymer chains or dissolved air molecules undergo unwanted interactions that might disrupt hydration. These beakers are sealed tightly with parafilm to create an airtight enclosure, which prevents moisture loss, reduces oxidative exchange, and maintains sterility throughout the hydration period. Because no Biochanin-A is present, the vehicle hydrogel offers a clear matrix in which polymer hydration, chain swelling, and three-dimensional network formation can be observed without interference from hydrophobic particulate matter. After sealing, the beakers are placed inside a refrigeration unit and left undisturbed for 48 hours. The absence of agitation during refrigeration is important because the polymer network forms through slow, uniform chain entanglement that progresses optimally under quiescent conditions. Agitation during this stage would interrupt network formation and potentially introduce air pockets or microstructural irregularities.

By ensuring identical hydration conditions as those used for the drug-loaded hydrogel, the vehicle hydrogel becomes an essential control that validates the technical effect of Biochanin-A incorporation. This embodiment thus enhances the robustness of the overall process by enabling comparative analysis of polymer performance, demonstrating reproducible handling characteristics, and establishing a scientific basis for distinguishing the functional impact of drug loading. The uniform rotational motion maintained during initial dispersion also ensures that both hydrogels-drug-loaded and drug-free-share an equivalent starting microstructure, which is necessary to support claims relating to improved physical properties, increased stability, or enhanced drug delivery characteristics of the Biochanin-A loaded hydrogel.

In an embodiment, the refrigerated storage facilitates completion of hydrogel formation through HPMC chain swelling, intermolecular hydrogen bonding, and aqueous entanglement, such that the final gel exhibits a semi-solid consistency attributable solely to the physicochemical interactions developed during the 48-hour storage period, wherein the addition of Biochanin-A is performed only after the initial HPMC hydration and dispersion are complete, and wherein the hydration of the hydroxypropyl methyl cellulose during the dispersion is further controlled by allowing an initial wetting period after the first portion of HPMC is added, such that the polymer granules undergo a preliminary phase of surface hydration before the next portion is introduced, and wherein the stirring speed is deliberately modulated in two intervals-first, a low-speed agitation of 100-200 rpm to permit air displacement from polymer interstices, followed by a moderate-speed agitation of 300-500 rpm to promote uniform chain expansion.

In this embodiment, the refrigerated storage plays a critical functional role in completing hydrogel formation by enabling hydroxypropyl methyl cellulose (HPMC) chains to undergo full swelling, intermolecular hydrogen bonding, and aqueous entanglement under controlled low-temperature conditions. When the hydrogel precursor is cooled between 2° C. and 8° C. without disturbance, the mobility of water molecules decreases gradually, allowing them to penetrate the polymer network in a uniform and sustained manner. This slow hydration mechanism supports the development of a cohesive three-dimensional gel matrix in which polymer chains expand, realign, and entangle. The final semi-solid consistency of the hydrogel is therefore a direct consequence of these physicochemical interactions rather than any external crosslinking agents or thermal activation, confirming that the material properties arise inherently from the interplay between HPMC and the aqueous medium during the 48-hour quiescent storage period.

The embodiment additionally specifies that Biochanin-A is added only after initial HPMC hydration and dispersion are complete. This sequencing is technically necessary because premature introduction of Biochanin-A would interfere with polymer swelling by decreasing water availability and introducing hydrophobic regions that promote agglomeration rather than uniform dispersion. By first creating a stable hydrated polymer environment with defined viscosity and dispersion characteristics, the system ensures that Biochanin-A is effectively incorporated into the matrix without clumping or undergoing sedimentation. The partially hydrated polymer acts as a suspending medium, allowing Biochanin-A molecules to become entrapped within the forming network through hydrogen bonding and steric encapsulation mechanisms.

Control over HPMC hydration is further enhanced by allowing an initial wetting period after the first portion of polymer is added to the aqueous phase. During this period, the granules partially swell at their surfaces, transitioning from dry particles into hydrated domains that can more readily accept subsequent polymer additions. This staged hydration prevents the formation of dry lumps or dense agglomerates, which would otherwise impede uniform chain expansion. The approach ensures that each successive portion of HPMC is introduced into a medium already capable of dispersing and hydrating polymer uniformly.

Moreover, the stirring speed is intentionally modulated in two deliberate intervals to optimize polymer hydration dynamics. In the first interval, low-speed agitation of 100-200 rpm gently moves the aqueous medium around the hydrating HPMC particles, enabling trapped air within the polymer interstices to escape. This air-displacement step is essential because entrapped air pockets can prevent full hydration of polymer particles, leading to micro-voids and weakened gel structure. In the second interval, the speed is increased to a moderate 300-500 rpm to supply sufficient mechanical shear for uniform chain expansion and dispersion. This controlled escalation of stirring intensity ensures that hydration proceeds efficiently without generating excessive shear heat that could prematurely increase viscosity or destabilize the polymer structure.

Together, these coordinated steps-sequencing drug addition after initial polymer hydration, allowing preliminary wetting of polymer granules, and modulating stirring speeds-create a synergistic and technically robust process that reproducibly yields a stable, semi-solid Biochanin-A hydrogel. The embodiment therefore fully supports the claimed invention by demonstrating how each procedural feature contributes to predictable gel formation and uniform drug incorporation.

In an embodiment, the introduction of Biochanin-A into the hydrated polymer dispersion is further regulated by adjusting the dispersion environment to minimize hydrophobic agglomeration of Biochanin-A particles, such that the addition occurs simultaneously with controlled vortex depth adjustment on the stirrer and optional pre-wetting of Biochanin-A with a minimal aliquot of distilled water to prevent particle flotation, and wherein the mixture is intermittently scraped from the beaker walls using a chemically inert spatula to prevent undissolved Biochanin-A from adhering to the vessel boundary surfaces, and wherein the mixing and dissolution are performed within an enclosed laboratory environment controlled for airborne particulate contamination, and wherein the containers used for hydration are pre-rinsed with distilled water and dried under a laminar airflow cabinet, and further wherein parafilm sealing is applied in multiple overlapping layers to provide a semi-hermetic barrier that restricts ingress of moisture vapor and airborne microorganisms during the 48-hour hydration period.

In this embodiment, the introduction of Biochanin-A into the hydrated HPMC dispersion is controlled with deliberate adjustments to the dispersion environment to mitigate the natural tendency of the hydrophobic Biochanin-A particles to agglomerate upon contact with an aqueous medium. By synchronizing the addition of Biochanin-A with a controlled adjustment of the vortex depth generated by the stirrer, the process ensures that each aliquot of Biochanin-A is immediately drawn into a turbulent yet downward-flowing zone of the dispersion. A deeper vortex facilitates rapid interior incorporation of the powder, whereas a shallower vortex reduces splashing and airborne dispersion of fine particles. This dynamic control enables optimal wetting and prevents the powder from floating on the surface, which is a common challenge when working with hydrophobic phytochemicals. The process may optionally include a pre-wetting step, wherein Biochanin-A is moistened with a minimal aliquot of distilled water before addition. Pre-wetting reduces particle buoyancy and facilitates immediate hydration at the polymer-water interface, thereby minimizing agglomeration and sedimentation and improving overall dispersion efficiency.

To further ensure uniform incorporation, the operator periodically scrapes the beaker walls using a chemically inert spatula, such as one made from PTFE. Hydrophobic compounds, particularly flavonoids like Biochanin-A, often exhibit adherence to the glass surfaces of reaction vessels. Without intermittent scraping, these adhered residues would remain undispersed, leading to inconsistencies in concentration and reduced dosing accuracy.

Removing these residues ensures that all material becomes available for incorporation into the polymer matrix and that the final hydrogel contains a homogeneous distribution of active compound.

The mixing and dissolution operations are performed within an enclosed laboratory environment that is controlled for airborne particulate contamination. This environmental control prevents extraneous dust, microbial spores, and particulate matter from entering the hydrogel precursor during the dynamic mixing phase, when the system is most vulnerable to exposure due to open-vessel stirring. Maintaining a clean environment is essential for the reproducibility and shelf-stability of hydrogels intended for biomedical or cosmetic use, as even trace contaminants can alter pH, viscosity, or microbial stability.

In addition, the containers used for the subsequent hydration period are pre-rinsed with distilled water and dried under a laminar airflow cabinet. This preparation ensures that no residual ions, detergents, or particulates remain on the container surfaces, which could otherwise interfere with HPMC hydration, destabilize Biochanin-A, or introduce microheterogeneities in the gel network. Drying under laminar flow preserves sterility and prevents airborne contaminants from settling onto the container walls before the hydrogel precursor is transferred.

Finally, sealing with parafilm is performed in multiple overlapping layers to create a semi-hermetic barrier around the container opening. Unlike a single thin layer, multi-layer sealing provides enhanced resistance to moisture vapor transfer, prevents evaporative thickening of the hydrogel, and restricts ingress of airborne microorganisms during the 48-hour hydration period. This semi-hermetic environment enables the HPMC chains to undergo complete swelling and network formation without external disturbance while simultaneously preserving the chemical stability of Biochanin-A by limiting oxidation and environmental exposure.

Overall, the embodiment establishes a technically rigorous set of conditions that ensure uniform drug dispersion, controlled hydration kinetics, and maintenance of sterility-all of which are essential for producing a high-quality Biochanin-A hydrogel with consistent physicochemical properties.

In an embodiment, the refrigerated storage period is not limited to passive cooling but further comprises allowing the hydrogel to undergo staged viscosity maturation characterized by polymer chain realignment under low-temperature quiescent conditions, such that the first 12 hours permit the onset of initial thickening of the hydrogel network, the subsequent 12 hours promote completion of HPMC chain swelling, and the final 24 hours allow molecular-level reorganization of the polymeric matrix to yield a structurally coherent hydrogel exhibiting uniform macroscopic consistency suitable for subsequent formulation handling, and wherein the Biochanin-A powder is optionally subjected to a preliminary screening comprising passing the powder through a 60-120 mesh stainless-steel sieve prior to dispersion, such that oversized agglomerates or compacted lumps are removed.

In this embodiment, the refrigerated storage period is described not merely as a passive cooling step but as an active and essential phase in which the hydrogel undergoes staged viscosity maturation driven by controlled polymer chain dynamics under low-temperature, quiescent conditions. When the Biochanin-A-HPMC dispersion is placed in refrigeration between 2° C. and 8° C., the reduced thermal energy environment slows molecular motion and allows the hydration, swelling, and alignment of HPMC chains to proceed in a highly ordered and time-dependent manner. During the first 12 hours of storage, the polymer network enters an initial thickening stage in which water penetrates the outer surfaces of the HPMC granules and begins to induce chain loosening and early-stage entanglement. This results in a measurable increase in viscosity, marking the transition from a fluid dispersion to a proto-gel state. The subsequent 12-hour interval supports the completion of polymer swelling, during which water molecules continue diffusing into the internal regions of each polymer chain. This extended swelling period is necessary because HPMC hydration is diffusion-controlled and proceeds gradually at low temperatures. At this stage, the polymer chains expand fully and begin interacting through intermolecular hydrogen bonding, giving rise to a semi-solid network.

The final 24 hours of refrigerated storage enable molecular-level reorganization of the polymeric matrix. During this period, the swollen HPMC chains rearrange into a stable three-dimensional configuration through sustained hydrogen bonding and physical entanglement. This quiescent structural optimization results in the formation of a coherent hydrogel exhibiting uniform macroscopic consistency and predictable rheological behavior. Such a staged maturation process is critical for producing a hydrogel suitable for downstream formulation handling, including spreading, syringe filling, or incorporation into composite dosage forms. Without this controlled, time-dependent maturation, the gel may remain heterogeneous, contain zones of incomplete hydration, or exhibit unstable viscosity during use.

Additionally, this embodiment provides that Biochanin-A powder may undergo preliminary screening by sieving through a stainless-steel mesh of 60-120 gauge prior to dispersion. This pre-processing step removes oversized agglomerates or compacted lumps that could otherwise resist dissolution or hydration during mixing. By ensuring that only fine, uniform particles enter the dispersion medium, the sieving step enhances wetting efficiency, reduces the risk of sedimentation, and improves homogeneity of drug distribution within the maturing hydrogel matrix. In practice, hydrophobic phytochemical powders like Biochanin-A commonly contain cohesive clusters that do not disperse under moderate shear; removing these ensures smoother incorporation, more predictable release characteristics, and improved reproducibility across batches.

Overall, the embodiment demonstrates that refrigeration provides not only cooling but also the ideal environment for controlled polymer network development, while optional pre-sieving of Biochanin-A enhances compatibility between the drug and the hydrophilic polymer. Together, these features contribute synergistically to the formation of a structurally stable, homogeneous hydrogel with reliable performance characteristics.

In an embodiment, the mechanical stirring is regulated to avoid excessive shear forces that could induce premature thermal warming of the dispersion fluid, such that the stirring device is operated below its heating threshold, and wherein the stirring rod, if used, is fabricated from a non-metallic, thermally non-conductive material to prevent localized hot spots within the dispersion, thereby maintaining the entire hydrogel-forming environment below 30° C. to prevent any structural alteration of Biochanin-A or destabilization of the polymer hydration process, and wherein the distilled water used as the aqueous medium is pre-degassed by heating to 60-70° C. followed by cooling to ambient temperature or by subjecting the water to vacuum degassing for at least 10 minutes.

In this embodiment, the mechanical stirring conditions are carefully regulated to prevent the generation of excessive shear forces that could inadvertently raise the temperature of the dispersion fluid. Stirring-induced thermal elevation is a well-recognized phenomenon in viscous polymer systems, particularly during prolonged mixing. To avoid such warming, the stirring device is operated strictly below its heating threshold, ensuring that no incidental thermal transfer occurs from the instrument's motor or magnetic coupling. This control is essential because both Biochanin-A and HPMC exhibit temperature-sensitive behaviors: Biochanin-A may undergo undesirable structural alteration or oxidative degradation above certain temperatures, and HPMC possesses thermogelation properties that cause premature viscosity increase or localized gelling when temperatures exceed approximately 30° C. Maintaining the entire hydrogel-forming environment below this threshold therefore preserves chemical integrity and ensures that hydration proceeds uniformly rather than through temperature-triggered phase transitions.

If a stirring rod is used instead of or in addition to a magnetic stir bar, the rod is fabricated from a non-metallic, thermally non-conductive material such as PTFE or high-grade polymer. These materials do not transfer heat to the surrounding dispersion, preventing the formation of localized hot spots that could compromise the hydration behavior of HPMC or the stability of Biochanin-A. Metallic rods, in contrast, may conduct heat from the stirrer's drive system, causing small but meaningful deviations in local temperature, which can influence polymer swelling kinetics and drug stability. The use of non-conductive materials thus ensures a homogeneous thermal environment throughout the mixing process.

The embodiment further specifies that the distilled water employed as the aqueous medium is pre-degassed prior to polymer dispersion. Degassing may be accomplished by heating the water to 60-70° C. and subsequently allowing it to cool to ambient temperature, or alternatively by subjecting the water to vacuum degassing for at least ten minutes. Removing dissolved gases serves several technical purposes: it reduces foaming during stirring, prevents the trapping of air bubbles within the forming hydrogel network, and promotes more complete hydration of HPMC granules by eliminating micro-bubbles that might otherwise lodge between polymer strands. Entrained air is known to disrupt uniform polymer swelling and can lead to heterogeneous gel density or microcavitation defects. Degassed water therefore supports the formation of a smoother and more uniform gel matrix.

Collectively, this embodiment ensures that the hydrogel-forming system remains chemically stable, thermally consistent, and free of structural imperfections caused by shear-induced heating or trapped gases. By maintaining low-temperature conditions, using thermally neutral stirring components, and preparing degassed water, the process minimizes variables that could compromise gel uniformity or Biochanin-A integrity, thereby supporting the reproducibility and technical reliability of the final hydrogel formulation.

In an embodiment, following Biochanin-A incorporation and prior to sealing, the mixture is allowed to stand undisturbed for a predetermined period of 5-10 minutes to permit natural gravitational settling of transient air pockets introduced during mechanical agitation, and wherein the container is gently tapped to assist bubble migration, and wherein the container used for hydrogel hydration is formed of borosilicate glass possessing low surface reactivity, and wherein the internal walls of the container are optionally coated with a thin film of distilled water prior to transfer of the hydrogel precursor to minimize polymer adhesion, aggregation at the vessel boundaries, or uneven polymer distribution.

In this embodiment, once Biochanin-A has been fully incorporated into the hydrated HPMC dispersion, the mixture is allowed to rest in a quiescent state for a period of approximately 5-10 minutes before sealing. This resting interval serves a critical technical function: it enables transient air pockets introduced during mechanical stirring to rise naturally to the surface under gravitational forces. Air entrainment is common in polymeric dispersions, especially when stirring occurs at moderate to high speeds, and the presence of these air pockets can lead to structural inconsistencies within the final gel network. By pausing the process and allowing the mixture to remain undisturbed, these bubbles are given sufficient time to migrate upward and dissipate. To further facilitate this process, the operator may gently tap the container, which helps dislodge microbubbles clinging to the container walls or suspended within the viscous medium. The removal of entrapped air is essential for ensuring uniform gel density, preventing microcavities in the hydrogel, and supporting reproducibility across batches.

The embodiment further specifies that the container used for hydrogel hydration is constructed from borosilicate glass, a material chosen for its low surface reactivity, high chemical resistance, and minimal leaching characteristics. Borosilicate glass provides a stable and inert environment for the hydration process, ensuring that neither the polymer nor Biochanin-A interacts with the container surface in a manner that could alter the hydrogel's physical or chemical properties. The smooth, non-porous interior of borosilicate glass also reduces the likelihood of polymer chains anchoring to surface irregularities, thereby promoting homogeneous network formation throughout the entire volume of the hydrogel.

To further enhance dispersion uniformity, the internal walls of the container may optionally be coated with a thin film of distilled water before the hydrogel precursor is transferred. This pre-wetting step reduces surface tension between the forming hydrogel and the glass vessel, thereby minimizing the adhesion of concentrated polymer aggregates along the walls. Without such pre-wetting, early hydration layers can cling to the container surface, resulting in localized thickening, uneven polymer distribution, or stagnation zones in which hydration kinetics differ from the bulk phase. Coating the walls with water allows the gel precursor to slide uniformly into position, ensuring that polymer chains maintain consistent contact with the dispersing medium and that the ensuing hydration proceeds evenly across the entire container volume.

Together, the steps in this embodiment ensure that the hydrogel precursor enters the 48-hour refrigeration phase in an optimized, air-free, and uniformly distributed condition. By eliminating trapped bubbles, preventing polymer adhesion, and providing an inert hydration environment, the embodiment supports the formation of a cohesive, defect-free hydrogel matrix and enhances the overall technical reliability of the process.

In an embodiment, the distilled water medium may optionally be pre-adjusted to a mildly acidic or neutral pH between 5.8 and 7.0 using dilute acid or base to optimize HPMC hydration behavior, and wherein the pH is monitored using a calibrated glass-electrode pH meter prior to polymer addition, with the conditioning improving polymer chain expansion and reducing hydration latency during the stirring process, and wherein the hydrogel precursor is transferred to refrigeration under a controlled cooling rate such that the temperature decreases gradually rather than abruptly, and wherein the cooling profile is achieved by placing the container first at ambient temperature inside the refrigerator door compartment and subsequently moving it deeper into the refrigeration chamber after 2-3 hours.

In this embodiment, the distilled water used as the hydration medium is optionally pre-conditioned to a mildly acidic or neutral pH range of 5.8 to 7.0 in order to optimize the hydration kinetics and chain expansion behavior of hydroxypropyl methyl cellulose (HPMC). HPMC exhibits pH-sensitive hydration characteristics, with polymer swelling and chain relaxation occurring more efficiently in this mildly acidic to neutral range. Adjusting the water to this pH prior to polymer addition ensures that the hydration environment is chemically aligned with the conditions under which HPMC demonstrates maximal solubility and dispersion uniformity. The adjustment is performed using dilute acid or base solutions, and the final pH is verified using a calibrated glass-electrode pH meter to ensure accuracy and reproducibility. This pre-conditioning step reduces hydration latency during stirring, meaning that polymer granules hydrate more quickly and uniformly, producing a smoother dispersion and reducing the risk of clumping or incomplete wetting during early-stage mixing.

Following dispersion, the hydrogel precursor is transferred to refrigeration under a controlled cooling rate rather than being subjected to sudden temperature drop. A gradual cooling profile is technically advantageous because it prevents rapid viscosity changes that may lead to microstructural defects within the forming hydrogel matrix. Sudden cooling can cause abrupt polymer contraction or uneven swelling, which may disrupt chain entanglement and create zones of inconsistent gel density. To achieve this controlled thermal transition, the sealed container is first placed at ambient temperature within the door compartment of the refrigerator, a region that experiences a more moderate temperature gradient. After an initial period of 2-3 hours, during which the temperature decreases gradually, the container is moved deeper into the refrigeration chamber—typically the coldest region—where it remains for the remainder of the 48-hour maturation period.

This gradual transition from ambient temperature to full refrigeration enables uniform polymer reorganization and ensures that Biochanin-A remains evenly distributed throughout the matrix as viscosity progressively increases. By combining pH conditioning with a controlled cooling profile, this embodiment enhances the efficiency of HPMC hydration, promotes consistent gel formation, and minimizes internal stresses that could otherwise lead to phase separation or non-uniform rheological behavior. The embodiment thus contributes significantly to the predictability and reproducibility of the hydrogel formation process by establishing optimal chemical and thermal environments during critical early stages of gel development.

In an embodiment, the hydrogel precursor, immediately prior to sealing, is subjected to a preliminary macroscopic homogeneity check performed under white light illumination, wherein the operator visually evaluates the dispersion for color uniformity, absence of particulate clusters, consistency of opacity, and smoothness of the polymer phase, and wherein the mixture is re-stirred for a brief period if any visible heterogeneity is detected.

In this embodiment, the hydrogel precursor undergoes a macroscopic homogeneity assessment immediately prior to the sealing step to ensure that the dispersion entering the 48-hour refrigeration phase is structurally uniform and free from visible defects that could compromise final gel quality. The evaluation is performed under neutral white light illumination, which provides consistent color rendering and enhances the operator's ability to detect subtle variations in dispersion quality. During this inspection, the operator visually examines the mixture for color uniformity, as Biochanin-A imparts a characteristic hue that should be evenly distributed throughout the hydrated HPMC matrix if proper dissolution and incorporation have occurred. Any deviation in color intensity or gradient indicates localized aggregation of Biochanin-A or incomplete mixing.

The operator also checks for the absence of particulate clusters, which may arise from insufficient hydration of HPMC granules, incomplete dispersion of Biochanin-A, or formation of microagglomerates during stirring. Such clusters, if not removed or redistributed, can create inconsistencies in drug loading, lead to differential swelling behavior, or disrupt the mechanical uniformity of the resulting hydrogel. The consistency of opacity is similarly evaluated; a uniform semi-translucent or opaque appearance suggests that polymer hydration has progressed evenly, whereas streaks, cloudiness, or stratification may signal regions of incomplete chain expansion or settling.

The smoothness of the polymer phase is another important visual parameter. A well-hydrated HPMC dispersion should exhibit a cohesive, uninterrupted appearance without granular residues or surface irregularities. If any heterogeneity is detected through this visual inspection, the mixture is re-stirred briefly to correct the inconsistency before sealing. This corrective stirring ensures that all particles—both polymer and Biochanin-A—are fully integrated into the dispersion, allowing the hydration and maturation processes that occur during refrigeration to proceed under optimal conditions.

By incorporating this homogeneity check, the embodiment introduces a quality assurance step that increases batch reliability, ensures consistent gel performance, and minimizes the likelihood of defects in the final hydrogel structure. This visual inspection step therefore plays a critical enabling role in ensuring that the hydrogel precursor begins its maturation phase in a fully uniform state, reinforcing the technical reproducibility of the process.

In an embodiment, during the weighing stage the Biochanin-A and hydroxypropyl methyl cellulose are equilibrated to ambient laboratory temperature for at least ten minutes before measurement so that temperature-related mass fluctuations are minimized, and wherein both materials are handled using antistatic weighing paper to avoid electrostatic retention of fine powder during transfer to the mixing vessel, and wherein during the polymer-dispersion stage the distilled water is allowed to rest motionless in the vessel long enough for thermal stabilization before stirring begins, after which the stirring speed is incrementally raised from an initial 150 rpm to a speed in the range of 350-450 rpm, producing a vortex with a depth not greater than one-third of the total liquid depth to allow inward draw of the first portion of hydroxypropyl methyl cellulose.

In this embodiment, the weighing stage is conducted with specific controls to ensure precise mass measurement and consistent material handling, both of which are essential for reproducibility in hydrogel formulation. Biochanin-A and hydroxypropyl methyl cellulose (HPMC) are first equilibrated to ambient laboratory temperature for at least ten minutes before measurement. This equilibration period prevents temperature-related mass fluctuations caused by transient thermal expansion or contraction of the powders, as well as density variations that can alter the balance reading. Materials weighed immediately after removal from cold storage or warm environments often exhibit drifting mass values due to condensation or convection currents; allowing them to stabilize at room temperature eliminates these sources of error and ensures accurate dosing of both the active ingredient and polymer.

To further improve handling precision, both Biochanin-A and HPMC are transferred using antistatic weighing paper. Fine powders, particularly organic compounds like Biochanin-A, tend to accumulate static charge that causes particles to cling to surfaces and be lost during transfer. Antistatic weighing paper prevents electrostatic retention, ensuring that the entire measured amount reaches the mixing vessel and that the final formulation matches the intended drug-to-polymer ratio. Accurate transfer is essential for maintaining controlled viscosity, drug loading efficiency, and the physicochemical properties of the final hydrogel.

During the polymer-dispersion stage, the distilled water used as the hydration medium is allowed to rest motionless in the mixing vessel long enough to achieve thermal stabilization. This step removes thermal gradients that could influence the initial hydration behavior of HPMC, since hydration rates vary with even slight temperature differences within the liquid column. Once stabilized, stirring begins at an initial speed of 150 rpm, generating gentle fluid motion that prevents splashing while preparing the system for polymer introduction. The stirring speed is then gradually increased to the range of 350-450 rpm to create a controlled vortex with a depth not exceeding one-third of the total liquid depth. Maintaining this vortex depth is critical: it ensures that the first portion of HPMC introduced is drawn inward and downward into the bulk fluid rather than floating on the surface, yet the vortex is not so deep that it entrains excessive air into the mixture.

This controlled introduction of HPMC allows the polymer granules to hydrate evenly and prevents premature clumping or dry-pocket formation that commonly occurs when powder is added to stagnant or insufficiently mixed water. The gradual increase in stirring speed also reduces mechanical stress on the polymer chains and avoids sudden shear forces that might disrupt early hydration dynamics. Together, these controlled steps-temperature equilibration, antistatic handling, water stabilization, and vortex management-ensure predictable polymer dispersion and lay the foundation for a stable, uniform hydrogel network to form in subsequent stages of the process.

In an embodiment, during introduction of hydroxypropyl methyl cellulose the powder is dispensed through a narrow-gauge funnel positioned above the fluid vortex so that the polymer contacts the aqueous medium in a dispersed pattern rather than in clumps, and wherein the operator varies the angle of the funnel by small arcs to distribute the powder across the central region of the rotating water column, and wherein the polymer-dispersion stage includes intermittent slowing of the rotational speed for visual assessment of polymer distribution, followed by resumption of higher speeds to promote deeper hydration, and wherein each slowdown event does not exceed ten seconds so that partially hydrated polymer does not settle on the vessel bottom.

In this embodiment, the introduction of hydroxypropyl methyl cellulose (HPMC) into the aqueous medium is carried out with a high degree of control to ensure that the polymer disperses uniformly and does not form clumps, which are difficult to hydrate fully and can compromise the structural integrity of the final hydrogel. To achieve this, the HPMC powder is dispensed through a narrow-gauge funnel positioned directly above the vortex generated in the rotating water column. The narrow opening of the funnel ensures that the polymer enters the fluid in a fine, controlled stream rather than as large aggregates. As the powder descends into the vortex, it encounters a zone of enhanced shear, which promotes rapid wetting of the outer surfaces of the granules and reduces surface agglomeration. This dispersed contact pattern is essential for consistent swelling and hydration of the polymer.

To further improve distribution, the operator varies the angle of the funnel by gently sweeping it in small arcs over the central region of the vortex. This controlled motion introduces the polymer across multiple entry points, preventing localized overloading of HPMC in any single region of the water column. Such variation in powder deposition ensures that hydration is not spatially restricted and that the polymer chains hydrate more evenly throughout the early dispersion phase. This is particularly beneficial because HPMC tends to form gelatinous outer layers upon initial wetting; distributing the polymer over a broader region ensures that these layers do not converge prematurely and form lumps.

The polymer-dispersion stage also includes intermittent slowing of the stirrer's rotational speed to allow the operator to visually assess the progress of polymer distribution. During these brief pauses, the operator can evaluate whether any undispersed polymer remains floating on the surface or if clumps are forming along the vessel walls. Importantly, each slowdown event is limited to no more than ten seconds. This time limit prevents partially hydrated polymer granules from settling to the bottom of the vessel, where they would be shielded from adequate fluid flow and become more difficult to incorporate uniformly into the dispersion.

After each brief assessment, the stirring speed is resumed to its higher range, restoring the vortex needed to promote deeper hydration of the polymer chains. This alternating cycle of brief inspection and renewed mixing allows for dynamic quality control without compromising hydration kinetics. The stirring-pause-stirring pattern facilitates early detection of inconsistencies while ensuring that the hydration process remains uninterrupted enough to produce a smooth, uniform polymer dispersion.

By combining controlled powder introduction, strategic funnel positioning, dynamic funnel angling, and timed speed modulation, this embodiment ensures optimal hydration behavior of HPMC and mitigates risks of clumping, sedimentation, or localized gelation. These process refinements directly contribute to the reproducibility and structural uniformity of the final Biochanin-A-loaded hydrogel.

In an embodiment, the polymer-dispersion stage is conducted in a vessel having a rounded internal base profile that promotes continuous fluid circulation and restricts the formation of stagnant hydration zones along internal corners, and wherein the vessel is rotated slightly by hand to modify gravitational flow during mixing, and wherein prior to Biochanin-A addition the hydrated polymer is subjected to a brief collapse of the vortex by lifting the stirrer vertically, followed by reinsertion of the stirrer, so that microbubbles introduced during the hydration phase move toward the surface and reduce disruption of the dispersion field during subsequent addition of Biochanin-A.

In this embodiment, the polymer-dispersion stage is intentionally carried out in a vessel designed with a rounded internal base profile, such as a hemispherical or smoothly curved-bottom beaker, which facilitates continuous and uniform fluid circulation during stirring. Unlike flat-bottom vessels that create angular stagnation zones where polymer granules can accumulate and hydrate unevenly, a rounded base prevents the formation of such dead zones by guiding the rotating fluid in uninterrupted circular paths. This design ensures that all HPMC particles experience similar shear forces and hydration conditions, resulting in uniform polymer swelling and reducing the likelihood of localized clumping or incomplete dispersion.

To further enhance the uniformity of fluid flow, the operator may gently rotate the vessel by hand during the early hydration stage. This slight manual rotation modifies gravitational flow patterns inside the vessel, breaking up potential symmetry in the vortex and allowing fluid to wash over different regions of the vessel walls. Such intervention prevents polymer from adhering to specific boundary areas and promotes more comprehensive hydration. The technique is especially effective in ensuring that fine particles that initially accumulate near the periphery of the vortex are drawn back into the main circulation path rather than forming semi-hydrated residues.

Before introducing Biochanin-A into the mixture, the embodiment specifies that the hydrated polymer undergoes a controlled collapse of the vortex. This is achieved by briefly lifting the stirrer vertically from its operational position, causing the vortex to dissipate and allowing the fluid surface to level momentarily. When the vortex collapses, microbubbles that were entrained during the hydration stage rise to the surface and escape from the dispersion. These microbubbles, if left within the vortex, could expand or accumulate during Biochanin-A addition, disrupting the dispersion field and causing irregular mixing behavior. After allowing these bubbles to escape, the stirrer is reinserted and mixing resumes, restoring a stable vortex suitable for consistent introduction of the active ingredient.

This sequence-using a rounded-base vessel, rotating the vessel for improved gravitational blending, and collapsing the vortex to remove microbubbles-ensures that the polymer base is in an optimal physical state before Biochanin-A addition. The preparation minimizes turbulence inconsistencies, enhances hydration uniformity, and ensures that the dispersion field is sufficiently stable for proper incorporation of the hydrophobic active compound. Collectively, these actions prevent structural defects in the developing hydrogel and contribute to a more predictable and homogeneous final formulation.

In an embodiment, during Biochanin-A incorporation each aliquot of Biochanin-A is directed toward a turbulent region of the rotating dispersion located between the center of the vortex and the mid-radius of the vessel so that the powder is drawn into a descending circulation path immediately upon contact with the hydrated polymer, and wherein the Biochanin-A incorporation stage includes temporarily reducing stirring speed by 20-50 rpm during each aliquot addition to promote initial wetting of Biochanin-A particles, followed by restoration of the stirring speed to re-establish the primary mixing regime needed for distribution of Biochanin-A within the polymer matrix.

In this embodiment, the incorporation of Biochanin-A into the hydrated HPMC dispersion is executed with precise spatial and dynamic control to ensure efficient wetting, rapid dispersion, and prevention of hydrophobic agglomeration. Each aliquot of Biochanin-A is deposited into a specifically targeted turbulent region of the rotating fluid-positioned between the central axis of the vortex and the mid-radius of the vessel. This region represents an optimal balance between shear intensity and downward flow: it is turbulent enough to disperse powder upon contact, yet sufficiently stable to direct the particles into a descending circulation stream rather than allowing them to float on the surface. By ensuring that each aliquot enters this zone, the process leverages the fluid dynamics of the system to draw Biochanin-A rapidly into deeper layers of the hydrated polymer, preventing surface accumulation or delayed wetting, both of which are common issues when incorporating hydrophobic flavonoids.

To further enhance the initial wetting efficiency, the stirring speed is deliberately reduced by approximately 20-50 rpm at the moment each aliquot of Biochanin-A is introduced. This temporary reduction minimizes splashing, decreases surface shear turbulence, and allows the hydrophobic particles to contact the aqueous polymer phase more gradually. The lowered speed decreases the likelihood that particles will be repelled by the fluid surface or dispersed as airborne powder. Instead, the Biochanin-A settles more smoothly into the vortex region where it begins hydrating and integrating with the polymer network. This controlled slowing also helps avoid sudden shear-related aggregation that can occur when powder is dropped into a high-energy fluid field.

Once the Biochanin-A has made initial contact with the hydrated polymer and wetting has commenced, the stirring speed is restored to its original higher rate. This re-establishes the primary mixing regime, generating sufficient turbulence, shear, and radial flow necessary to distribute the Biochanin-A uniformly throughout the matrix. The restored stirring conditions ensure that any partially hydrated particles are rapidly dispersed, minimizing the formation of local concentration gradients or sedimentation zones. By balancing lower-speed wetting with high-speed dispersion, the embodiment ensures that Biochanin-A is fully and consistently incorporated at both microscopic and macroscopic levels.

These process refinements-targeted powder placement, dynamic control of stirring speed, and strategic re-establishment of mixing conditions-collectively enhance the efficiency and homogeneity of Biochanin-A incorporation. They mitigate the intrinsic challenges posed by hydrophobic compounds and ensure that the active ingredient becomes uniformly integrated within the polymeric structure, thereby preventing inconsistencies in drug loading, texture, or release characteristics in the final hydrogel.

In an embodiment, during introduction of Biochanin-A a non-metallic spatula is used at intervals to detach adhering aggregates from the vessel walls so that undissolved particulate does not accumulate at boundary layers, and wherein the final homogenization period following Biochanin-A incorporation lasts not less than ten minutes with stirring conducted in a continuous uninterrupted mode to allow each polymer chain segment to interact with dissolved Biochanin-A molecules as the dispersion viscosity increases.

In this embodiment, the process accounts for the natural tendency of hydrophobic Biochanin-A particles to adhere to vessel walls during incorporation into the hydrated polymer matrix. To prevent the formation of persistent aggregates at the boundary surfaces, the operator periodically uses a non-metallic spatula-preferably fabricated from PTFE or another chemically inert, non-reactive material—to gently dislodge any adhering particulates. The use of non-metallic implements avoids scratching the vessel walls, prevents potential catalytic oxidation of Biochanin-A, and eliminates the risk of metal ion contamination, which could alter polymer hydration dynamics or drug stability. By clearing the boundary layers at regular intervals, the process ensures that all Biochanin-A material participates in the dispersion rather than remaining localized at the periphery, thereby promoting accurate drug loading and uniform distribution throughout the hydrogel matrix.

Following the incremental addition of Biochanin-A, the system enters a final homogenization period in which continuous, uninterrupted stirring is maintained for no less than ten minutes. This extended stirring duration is technically essential because, as the hydration of HPMC progresses, the viscosity of the dispersion gradually increases, thereby altering the shear environment and fluid dynamics within the vessel. Continuous stirring under these conditions ensures that Biochanin-A molecules are carried through the thickening polymer network long enough to interact with expanding polymer chain segments. These interactions include hydrogen bonding, hydrophobic association, and steric entrapment within the forming gel matrix. Allowing sufficient time for these molecular-level interactions supports the creation of a uniform drug-polymer distribution and prevents the emergence of microdomains with disproportionate Biochanin-A concentration.

This uninterrupted homogenization phase also enables the dispersion to transition smoothly into its pre-gel state without the risk of premature stratification or particle settling. Interrupting stirring during this stage could lead to uneven polymer hydration or localized separation of Biochanin-A, particularly because the dispersion has not yet reached the viscosity required to immobilize suspended particulates. By maintaining constant agitation, the process ensures that both polymer and drug components evolve together within the same dynamic environment, thereby producing a hydrogel precursor of consistent composition and predictable rheological behavior.

Overall, this embodiment contributes significantly to the robustness and reproducibility of the formulation process. The combination of boundary-layer control through strategic spatula use and extended homogenization enables complete incorporation of Biochanin-A, stable interaction with the hydrating polymer chains, and formation of a uniformly structured hydrogel precursor ready for final maturation during the refrigeration phase.

In an embodiment, the transfer stage includes lowering the stirrer speed gradually before switching it off so that the fluid motion decreases smoothly, and wherein the operator waits sufficient time for surface turbulence to dissipate before pouring the mixture into the receiving container, and wherein the container selected for receiving the hydrogel precursor is pre-cleaned by rinsing with distilled water and left to dry under low-turbulence airflow so that environmental particulates do not enter the hydrogel prior to sealing.

In this embodiment, the transfer stage is executed with deliberate control to preserve the uniformity and structural integrity of the hydrogel precursor immediately after homogenization. The stirrer speed is gradually lowered rather than stopped abruptly, allowing the rotational motion of the fluid to decelerate smoothly. This controlled reduction in agitation prevents the formation of residual vortices or abrupt shear gradients that could disturb the uniform distribution of Biochanin-A or cause partially hydrated HPMC strands to collapse irregularly. A sudden halt in stirring can create chaotic fluid motion or induce air entrainment, both of which may compromise the consistency of the precursor as it transitions to the static hydration phase.

Once the stirring device is fully stopped, the operator allows sufficient time—typically several minutes—for the surface turbulence to settle completely. This settling period ensures that any remaining micro-bubbles or suspended droplets migrate towards the surface and dissipate, leaving a stable, quiescent mixture. Pouring the precursor while turbulence remains could reintroduce air pockets, disturb particle distribution, or lead to inconsistent filling of the receiving container. The waiting time thus ensures that the hydrogel enters the refrigeration phase in an undisturbed, homogenized state conducive to reproducible polymer network formation.

The container selected for receiving the hydrogel precursor is pre-cleaned by thoroughly rinsing it with distilled water to remove any ionic contaminants, residues, or dust present on its surfaces. Distilled water is used specifically because it lacks dissolved salts or particulates that could interfere with the hydration chemistry of HPMC or alter Biochanin-A stability. After rinsing, the container is allowed to dry under low-turbulence airflow, such as inside a laminar flow cabinet or in a sheltered area free from circulating dust. This drying process ensures that no airborne particles settle inside the container prior to filling. Preventing environmental particulates from entering the system is crucial because the early hydration stages of the hydrogel precursor are highly sensitive to contamination; foreign particles can serve as unintended nucleation sites or alter local viscosity, leading to microstructural irregularities in the final gel.

Together, these steps ensure that the hydrogel precursor is transferred smoothly, without introducing defects, contaminants, or thermal or mechanical stresses that might compromise the reproducibility and performance of the final hydrogel. The gradual deceleration of stirring, the waiting period for turbulence dissipation, and the use of a pre-cleaned, particle-free container collectively contribute to a stable, consistent precursor ready for the subsequent sealing and refrigeration stages.

In an embodiment, the container interior is optionally moistened with a thin film of distilled water before pouring the hydrogel precursor so that polymer strands contacting the container wall do not undergo premature localized dehydration, wherein after pouring the hydrogel precursor into the container the fluid is left undisturbed for a settling interval of five to ten minutes so that entrained air bubbles migrate toward the surface and leave the bulk material free of internal cavities, wherein sealing of the container is accomplished by wrapping parafilm in multiple overlapping passes across the opening, applying light manual pressure at the rim to achieve close conformation of the sealing material, and extending the sealing material several centimeters down the neck region of the container, and wherein after sealing, the container is positioned initially in the upper region of the refrigeration compartment for gradual temperature descent, and after two to three hours it is relocated to the coldest region inside the refrigeration chamber to support the slow formation of the hydrogel network.

In this embodiment, the container intended to receive the hydrogel precursor is optionally moistened along its interior surface with a thin, uniform film of distilled water prior to filling. This pre-wetting serves an important functional purpose: it prevents premature localized dehydration of HPMC strands that come into contact with the vessel wall during transfer. Because HPMC begins to form a semi-gelled layer upon direct exposure to dry surfaces, pre-moistening ensures that polymer chains retain full access to the surrounding aqueous medium rather than adhering or partially drying at the boundary. This technique promotes uniform polymer swelling and prevents the development of dense, uneven layers along the container wall that could disrupt homogeneous hydration kinetics during the subsequent refrigeration period.

After the hydrogel precursor is poured into the prepared container, it is allowed to rest undisturbed for five to ten minutes. This settling interval provides time for entrained air bubbles—introduced during stirring or transfer—to rise toward the surface under gravitational forces. Removing these bubbles before sealing is essential, as internal air pockets can create structural voids that weaken the gel matrix, compromise its rheology, or interfere with controlled drug release properties. The quiescent settling phase ensures that the bulk of the material becomes free of internal cavities, supporting the formation of a uniform hydrogel network during refrigeration.

The sealing step is carried out using parafilm applied in multiple overlapping passes across the container opening. Each layer slightly overlaps the previous one, creating a robust, semi-hermetic seal that minimizes vapor loss while preventing ingress of airborne contaminants or microorganisms. Light manual pressure is applied around the rim to mold the parafilm closely to the container surface, ensuring a tight conformational fit. The seal is extended several centimeters down the neck region of the container, enhancing tensile strength and preventing accidental loosening during refrigeration. This layered sealing approach maintains the internal humidity and oxygen conditions necessary for consistent HPMC hydration and protects Biochanin-A from oxidation throughout the 48-hour maturation period.

Following sealing, the container is positioned initially in the upper region of the refrigeration compartment, where temperatures are slightly warmer and more stable. This transitional placement allows the hydrogel precursor to cool gradually rather than being abruptly exposed to the lowest temperatures in the chamber. Gradual cooling minimizes thermal shock, reduces sudden viscosity shifts, and prevents uneven polymer contraction. After two to three hours of controlled cooling, the container is relocated to the colder interior region of the refrigerator, where the temperature remains consistently between 2° C. and 8° C. In this lower-temperature environment, the hydrogel network forms slowly and uniformly as HPMC chains continue to swell, entangle, and align, giving rise to a cohesive, defect-free gel matrix.

By combining controlled pre-wetting, bubble removal, multi-layer parafilm sealing, and staged cooling, this embodiment ensures optimal conditions for hydrogel stabilization, structural uniformity, and reproducible drug distribution, thereby enhancing both technical reliability and formulation quality.

In an embodiment, the refrigeration stage is conducted without any vibration or movement of the container, and wherein the refrigeration shelf selected for storage is cleared of other items to avoid temperature cycling caused by door opening or airflow redirection, wherein the refrigerated hydrogel precursor undergoes progressive viscosity development across the forty-eight-hour storage period, including initial polymer swelling, followed by slow realignment of polymer chains, and culminating in formation of an internally cohesive gel phase, wherein the hydrogel precursor occupies no more than two-thirds of the container volume so that adequate headspace is available for thermal expansion of the aqueous medium during cooling.

In this embodiment, the refrigeration stage is carefully controlled to prevent disturbances that could interfere with the gradual and uniform formation of the hydrogel network. The container holding the hydrogel precursor is placed in a position within the refrigerator where it will remain free from vibration, movement, or mechanical disturbance throughout the entire 48-hour storage period. Vibrations—whether from frequent door opening, compressor cycling, or contact with other items—can disrupt early-stage polymer alignment, causing inconsistencies in gel density, localized thinning, or the introduction of internal shear patterns. By ensuring complete stillness during refrigeration, the system enables the polymer chains to swell, reorganize, and entangle in a stable, quiescent environment that fosters uniform gel formation.

To further support temperature stability, the shelf selected for storage is cleared of other items. Removing adjacent objects minimizes airflow disruption and reduces the likelihood of temperature fluctuations caused by door opening or air recirculation. Temperature cycling is known to significantly impact viscosity development in hydrophilic polymer systems, as repeated micro-shifts in temperature can cause alternating phases of polymer contraction and expansion. By isolating the hydrogel precursor on an uncluttered shelf, the formulation experiences a consistent cooling environment conducive to predictable viscosity evolution.

During the forty-eight-hour refrigeration period, the hydrogel precursor undergoes progressive, multi-stage viscosity development. In the initial phase, polymer swelling occurs as water continues to diffuse into the HPMC chains, loosening their structure and beginning the transition toward gelation. This is followed by a slow realignment phase in which the swollen chains gradually reorganize, forming intermolecular hydrogen bonds and physical entanglements characteristic of a maturing hydrogel network. In the final stage, these interactions culminate in the formation of a cohesive, semi-solid gel matrix that is structurally uniform and mechanically stable. This progressive development is dependent on maintaining an undisturbed environment throughout the maturation process; interruptions can halt or reverse chain alignment, creating defects that weaken the final gel.

The embodiment also specifies that the hydrogel precursor should occupy no more than two-thirds of the total container volume. Providing this headspace is necessary because the aqueous medium undergoes slight thermal expansion during the cooling process. Without sufficient overhead space, expanding fluid could exert pressure on the seal, potentially compromising the integrity of the parafilm barrier or causing overflow. Adequate headspace also prevents excessive compression of the forming gel, ensuring that polymer chains have room to extend and entangle without mechanical constraint. This volume-buffer approach contributes to the stability of both the gel structure and the sealed environment during the temperature descent.

Collectively, the stillness, temperature stability, staged viscosity development, and controlled volumetric headspace described in this embodiment ensure that the hydrogel matures into a robust, consistently structured material. These controlled conditions enhance reproducibility, prevent gel defects, and ensure the stable integration of Biochanin-A within the HPMC matrix.

In an embodiment, rotational mixing used in the Biochanin-A incorporation stage employs a stirrer blade angle selected to create vertical shear planes in addition to radial shear planes so that distribution of Biochanin-A occurs throughout the full depth of the fluid column, including the lower central region of the vessel where stagnation zones may otherwise occur, wherein the mixing vessel is positioned on a level surface verified by a bubble level so that the vortex axis remains vertical and does not drift laterally, which promotes symmetrical flow of the polymer and Biochanin-A during mixing, and wherein the first portion of HPMC introduced during dispersion is deliberately small, not exceeding 10% of the total polymer mass, so that the initial hydration phase forms a thin polymeric slurry that supports dispersion of the remaining polymer with reduced risk of agglomeration.

In this embodiment, the rotational mixing applied during the Biochanin-A incorporation stage is engineered with specific blade geometry to ensure complete three-dimensional dispersion of the hydrophobic active compound. The stirrer blade is oriented at an angle that generates not only radial shear-driving fluid outward from the center—but also vertical shear planes that draw material upward and downward within the vessel. This dual-shear configuration is essential for preventing stagnation zones, particularly in the lower central region of the vessel where hydrophobic powders may otherwise accumulate or escape adequate shear exposure. By promoting vigorous vertical circulation, the angled blade ensures that Biochanin-A particles descend rapidly into the fluid column, become submerged within the hydrated polymer phase, and undergo thorough wetting and disaggregation. This engineering choice directly enhances uniformity of drug distribution within the hydrogel matrix and minimizes the presence of localized concentration pockets.

To support symmetrical and balanced flow dynamics, the mixing vessel is positioned on a level laboratory surface verified with a bubble level. Ensuring that the vessel is perfectly horizontal prevents lateral drifting of the vortex axis, which could otherwise lead to uneven fluid motion, asymmetric mixing zones, and inconsistent powder incorporation. A tilted vessel causes the vortex to lean to one side, reducing shear forces in certain regions and creating dead zones where polymer or Biochanin-A may settle or hydrate inadequately. By maintaining a strictly vertical vortex axis, the system promotes consistent radial and vertical flow patterns throughout the entire mixing process, improving reproducibility and uniformity of the dispersion.

The embodiment further enhances hydration reliability by introducing only a small portion-no more than 10%—of the total HPMC mass at the start of the dispersion process. This initial fraction quickly hydrates to form a thin polymeric slurry, which provides a semi-viscous medium that improves subsequent dispersion of the remaining polymer. Without this preliminary slurry formation, adding the full polymer load into unconditioned water would result in high risk of clumping, as dry HPMC granules tend to form cohesive gelatinous shells upon first contact with water, trapping dry cores inside. By beginning with a small fraction, the system ensures that the first hydration phase produces a shear-responsive slurry that readily incorporates additional polymer without agglomeration. This controlled strategy greatly reduces hydration defects and ensures that the polymer network develops smoothly and uniformly.

By integrating vertical-radial shear blade geometry, ensuring precise vessel alignment, and utilizing staged polymer introduction, this embodiment provides a robust and technically optimized approach to both polymer hydration and Biochanin-A incorporation. These features collectively promote homogeneity, prevent stagnation, and enhance the structural integrity of the final hydrogel formulation.

Diabetes is accompanied with increased level of lipid peroxides and ROS and a decreased level of the key antioxidant enzymes SOD and CAT which play an important role in scavenging the toxic intermediate of incomplete oxidation. The diminished activity of these antioxidant enzymes results in the accumulation of superoxide anion and hydrogen peroxide within biological systems, subsequently triggering the generation of hydroxyl radicals that promote lipid peroxidation. SOD provides cellular protection against ROS through its scavenging activity, thereby preventing damage to membrane and biological structures. Specifically, this ubiquitous antioxidant enzyme catalyzes the dismutation of superoxide radical H₂O₂, which is subsequently converted to H₂O by CAT. Therefore, SOD functions as a primary defense mechanism against ROS and inhibits additional free radical formation. Studies have shown that SOD activity is diminished in diabetic conditions due to H₂O₂-mediated inactivation or enzyme glycation, which are commonly observed in diabetes. CAT plays a crucial role in H₂O₂ reduction, thereby protecting cells from highly reactive OH radicals. The findings demonstrate the potent antioxidant effects of BCA in diabetic wound healing. Analysis revealed significant elevations in the antioxidant enzymes SOD and CAT following BCA treatment. Supporting the observations from the present invention, a prior art reported similar enhancements in SOD and CAT activities when studying BCA effects in streptozotocin-induced diabetic rats. Hyperglycemia in diabetic patients induces increased free radical formation, leading to elevated oxidative stress and subsequent lipid peroxidation. MDA, which is a secondary product, has been proven to be cross-linked with protein and DNA, causing potential toxic effects. Furthermore, the aldehyde groups of MDA formed during lipid peroxidation act as an anchor between sugar and protein moieties, enhancing the formation of glycated proteins.

In the present invention, it is observed that diabetes significantly increased the lipid peroxidation product (MDA) levels. However, BCA treatment effectively decreased MDA levels in treated diabetic groups, demonstrating its antioxidant capacity. These findings confirm the BCA's ability to reduce MDA levels and enhance antioxidant defense mechanisms. Importantly, the investigation revealed that BCA's antioxidant effects operate through specific molecular pathways, evidenced by increased expression of transcription factor Nrf2 and NQO1 enzyme. The BCA's protective role against oxidative stress is demonstrated through Nrf2 pathway activation and NQO1 upregulation. The significance of these molecular mechanisms is further supported by a report where a comprehensive review documents the BCA's consistent antioxidant properties across various experimental models, confirming its therapeutic potential through the Nrf2/ARE pathway regulation.

Compared with nondiabetic wounds, diabetic wounds have a longer inflammatory phase of wound healing. This extended proinflammatory state delays wound healing and can lead to the formation of a chronic wound. The results demonstrated that BCA exhibits significant anti-inflammatory properties, as evidenced by the reduction in key inflammatory markers. Specifically, it is observed that a significant decrease in pro-inflammatory cytokines, including TNF-α, IL-1β, and IL-6, compared to the control group. These findings supported a previous work that reported BCA treatment effectively suppressed inflammatory markers, with significant reductions in IL-6, IL-1β, and TNF-α levels. Several signaling pathways linking diabetes with elevated COX-2 expression have been previously reported. In this context, the present investigation demonstrated a significant downregulation of COX-2 expression following BCA treatment, suggesting a direct effect on the inflammatory cascade. Furthermore, the results showed that BCA treatment led to significant inhibition of TLR4 levels compared to other groups. These findings complement previous studies showing that BCA possesses anti-inflammatory properties through inhibition of the TLR4 pathway.

BCA works by regulating the TLR4-associated signaling pathway, leading to reduced inflammatory response. A study confirmed that BCA alleviates inflammatory responses and inhibits TLR4 pathway activation in cells, reinforcing its role as a promising agent in regulating inflammatory response.

Late phases of wound healing involve proliferation and remodeling, where the growth and angiogenic factors and collagen deposition play a decisive role. In diabetic wounds, insufficient angiogenesis is one of the biggest contributors to poor wound healing, and occurs through several mechanisms. First, diabetic wounds have a deficit of necessary proangiogenic factors, possibly due to fewer macrophages that produce them. Additionally, antiangiogenic factors are upregulated, while capillary maturation factors are downregulated. In this regard, TGF-b family has been reported to play a crucial role in wound healing as it regulated fibrosis, inflammation and angiogenesis. The data indicated that BCA effectively modulates TGF-β1 signaling in diabetic wounds tissues. This regulatory effect was found to be consistent with recent studies showing that controlled TGF-β1 signaling is essential for proper wound healing. The modulation of this pathway by BCA appears to be particularly beneficial in the context of diabetic wound healing, where maintaining optimal TGF-β1 signaling levels is crucial for preventing impaired healing processes. Further, BCA exhibited pro-angiogenic properties in healing wounds as evidenced by increased expression of VEGFA and PDGFR (FIG. 11). This is in line with the known role of angiogenesis and angiogenic factors in wound healing. It has been reported that PDGFR-β signaling plays a fundamental role in fibroblast recruitment and activation during wound. Studies indicate that several isoflavone compounds play a pivotal role in promoting angiogenesis, demonstrated through their ability to increase the expression of VEGF and regulate its receptors VEGFR-2/Flk-1, leading to enhanced wound healing through the activation of cellular signaling pathways.

Remodeling is the last stage in wound healing, yet it is a crucial stage, and restoration of tissue integration may be delayed in diabetic wound conditions. In this framework, collagen deposition is advantageous during wound recovery. Studies have consistently shown that Biochanin A enhances Type I Collagen production in cells through increasing gene expression of essential extracellular matrix proteins, contributing directly to improved remodeling, particularly under conditions associated with oxidative stress. In the current study, Col 1A1 and Col 4A1 mRNA expression were enhanced by BCA (FIG. 12). Collagen type I is the most common type of collagens in healing skin tissues. In particular, Col 1A1 has been considered a reliable marker of collagen synthesis in the late stages of wound healing. Collagen IV plays essential roles in wound healing by promoting three crucial cellular processes: cell attachment, migration, and proliferation, which are fundamental for effective wound closure. Overall, the results demonstrated that BCA treatment efficiently enhanced wound healing activity in diabetic rats through multiple coordinated mechanisms. This was evidenced by the significant improvements in antioxidant defense systems through Nrf2/ARE pathway activation, marked reduction in inflammatory markers via TLR4 pathway modulation, enhanced angiogenic factors (VEGFA and PDGFR), and improved tissue remodeling through increased collagen synthesis. These findings were further confirmed by histological examinations which revealed that BCA-treated wounds exhibited enhanced healing with proper tissue regeneration and collagen deposition, suggesting BCA's promising therapeutic potential in diabetic wound management through its comprehensive effects on multiple healing pathways.

Diabetic wound healing represents a complex therapeutic challenge, where multiple factors, including oxidative stress, inflammation, and impaired angiogenesis, play crucial roles in delayed wound recovery. The present invention demonstrates the therapeutic potential of BCA in enhancing diabetic wound healing through multiple molecular mechanisms. BCA treatment exhibited remarkable wound healing properties through its comprehensive effects on various healing pathways. The therapeutic efficacy of BCA in promoting wound healing activity was evidenced at both tissue and molecular levels. The enhancement in wound healing can be attributed to its ability to combat oxidative stress through Nrf2/ARE pathway activation (increased SOD and CAT activities), modulate inflammation (reduced TNF-α, IL-1β, and IL-6 levels), and promote tissue regeneration (enhanced collagen synthesis and deposition). Additionally, BCA demonstrated significant pro-angiogenic properties as shown by the marked upregulation of VEGFA and PDGFR expression in diabetic rats, key factors essential for tissue regeneration during the wound healing process. Collectively, these findings underscore the therapeutic potential of BCA as a promising pharmacological agent for enhancing diabetic wound healing through its multi-targeted approach. These results provide a promising foundation for using BCA as a therapeutic agent in diabetic wound healing. However, several aspects require further investigation to establish their clinical efficacy. To better understand the mechanisms underlying the observed effects, further investigations are recommended. Exploring the intracellular pathways and molecular factors involved in the wound healing effects of BCA could provide valuable insights. This may involve the use of specific biomarkers or molecular analyses to dissect the healing mechanisms. Besides, it would be beneficial to perform dose-response experiments with BCA to determine the optimal concentration for wound healing activity. Testing a range of concentrations could help establish a clear dose-response relationship and determine the practicality of this compound for potential therapeutic interventions. While the presented results from the in vivo diabetic rat model are encouraging, it would be essential to further validate and expand upon these findings in additional relevant in vivo models or long-term assessments. This could help confirm the potential of BCA as a wound healing agent and its effects in more complex physiological settings. Managing diabetic wound healing presents several challenges due to its complex cellular responses and diverse healing stages. Therefore, different therapeutic approaches may be needed for different stages of wound healing and diabetes severity. The timing and duration of BCA administration may be crucial factors to consider in future clinical evaluations.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A process for preparing a Biochanin-A (BCA) loaded hydrogel, the process comprising:
weighing, using an analytical balance, Biochanin-A in an amount of 5 g and hydroxypropyl methyl cellulose (HPMC) in an amount of 1.5 g, wherein the HPMC is selected in a grade suitable for forming a semi-solid aqueous hydrogel matrix;
dispersing the 1.5 g of HPMC into 100 mL of distilled water maintained at room temperature by introducing the HPMC portion-wise under continuous mechanical agitation provided by a hotplate stirrer model LMS-1003, thereby forming an initial polymer suspension, wherein the stirring is carried out for a time sufficient to hydrate and uniformly wet the HPMC particulates;
continuing the stirring of the polymer suspension until the formation of a homogeneous viscous HPMC dispersion, the dispersion exhibiting no visible sedimentation or agglomeration, wherein the hydration of the polymer is carried out under ambient conditions without heating;
gradually adding the 5 g of Biochanin-A into the HPMC dispersion in incremental portions such that each aliquot is fully incorporated into the hydrating polymer matrix before the subsequent addition, and maintaining continuous stirring for a period of 10 minutes to facilitate dissolution and uniform distribution of Biochanin-A within the dispersion;
transferring the resulting Biochanin-A containing HPMC mixture into at least one container, sealing the container using parafilm to prevent evaporation or atmospheric contamination; and
subjecting the sealed mixture to refrigerated storage for a duration of 48 hours under conditions effective to allow complete polymer hydration, chain entanglement, and stabilization of the Biochanin-A loaded hydrogel structure,
wherein the distilled water used for dispersing HPMC has a conductivity below 10 μS/cm and is added in a single 100 mL volume prior to introducing the polymer, and wherein the dispersion comprises maintaining the water at 22-25° C. such that no thermal gelation of HPMC occurs, and wherein the dispersing of the HPMC comprises introducing the HPMC powder slowly into the central vortex of the mechanical stirrer to avoid clumping, and wherein the agitation speed is maintained sufficiently high to prevent the polymer from forming surface aggregates or undispersed globules, and wherein the Biochanin-A is added gradually into the hydrated HPMC matrix in incremental masses not exceeding 0.5 g at a time to enable dissolution within the viscous dispersion, and wherein the stirring is performed in a manner preventing sedimentation of Biochanin-A particulates within the hydrogel precursor, and wherein the introduction of Biochanin-A into the hydrated polymer dispersion is further regulated by adjusting the dispersion environment to minimize hydrophobic agglomeration of Biochanin-A particles, such that the addition occurs simultaneously with controlled vortex depth adjustment on the stirrer and optional pre-wetting of Biochanin-A with a minimal aliquot of distilled water to prevent particle flotation, and wherein the mixture is intermittently scraped from the beaker walls using a chemically inert spatula to prevent undissolved Biochanin-A from adhering to the vessel boundary surfaces, and wherein the mixing and dissolution are performed within an enclosed laboratory environment controlled for airborne particulate contamination, and wherein the containers used for hydration are pre-rinsed with distilled water and dried under a laminar airflow cabinet, and further wherein parafilm sealing is applied in multiple overlapping layers to provide a semi-hermetic barrier that restricts ingress of moisture vapor and airborne microorganisms during the 48-hour hydration period.

2. The process of claim 1, wherein the continuous stirring for dissolution of Biochanin-A is performed for at least 10 minutes without interruption, and wherein the resulting mixture achieves uniform macroscale distribution of Biochanin-A within the HPMC network prior to sealing, wherein sealing the hydrogel precursor with parafilm comprises covering the open surface of the container fully to prevent moisture exchange, oxidative exposure, and microbial ingress, thereby preserving the physicochemical stability of the hydrogel during the 48-hour hydration period, and wherein the refrigeration is carried out at a temperature between 2° C. and 8° C. for a minimum of 48 hours, such that full polymer hydration, viscosity stabilization, and network formation occur as the HPMC chains interact with aqueous media under low-temperature equilibration.

3. The process of claim 1, further comprising dispersing 1.5 g of HPMC in 100 mL of distilled water under identical stirring conditions, sealing the mixture, and refrigerating the hydrogel for 48 hours, wherein no Biochanin-A is added to said vehicle hydrogel, wherein the hydrogel precursor is stored in chemically inert laboratory glass beakers sealed with parafilm to provide an airtight enclosure, and wherein the beakers are placed in refrigeration without agitation to allow undisturbed polymer settling and full hydration, and wherein the stirring of HPMC and Biochanin-A is conducted using the LMS-1003 hotplate stirrer without activating the heating module, and wherein the stirrer maintains uniform rotational motion throughout the dispersion and dissolution stages.

4. The process of claim 1, wherein the refrigerated storage facilitates completion of hydrogel formation through HPMC chain swelling, intermolecular hydrogen bonding, and aqueous entanglement, such that the final gel exhibits a semi-solid consistency attributable solely to the physicochemical interactions developed during the 48-hour storage period, wherein the addition of Biochanin-A is performed only after the initial HPMC hydration and dispersion are complete, and wherein the hydration of the hydroxypropyl methyl cellulose during the dispersion is further controlled by allowing an initial wetting period after the first portion of HPMC is added, such that the polymer granules undergo a preliminary phase of surface hydration before the next portion is introduced, and wherein the stirring speed is deliberately modulated in two intervals-first, a low-speed agitation of 100-200 rpm to permit air displacement from polymer interstices, followed by a moderate-speed agitation of 300-500 rpm to promote uniform chain expansion.

5. The process of claim 1, wherein the refrigerated storage period is not limited to passive cooling but further comprises allowing the hydrogel to undergo staged viscosity maturation characterized by polymer chain realignment under low-temperature quiescent conditions, such that the first 12 hours permit the onset of initial thickening of the hydrogel network, the subsequent 12 hours promote completion of HPMC chain swelling, and the final 24 hours allow molecular-level reorganization of the polymeric matrix to yield a structurally coherent hydrogel exhibiting uniform macroscopic consistency suitable for subsequent formulation handling, and wherein the Biochanin-A powder is optionally subjected to a preliminary screening comprising passing the powder through a 60-120 mesh stainless-steel sieve prior to dispersion, such that oversized agglomerates or compacted lumps are removed.

6. The process of claim 1, wherein the mechanical stirring is regulated to avoid excessive shear forces that could induce premature thermal warming of the dispersion fluid, such that the stirring device is operated below its heating threshold, and wherein the stirring rod, if used, is fabricated from a non-metallic, thermally non-conductive material to prevent localized hot spots within the dispersion, thereby maintaining the entire hydrogel-forming environment below 30° C. to prevent any structural alteration of Biochanin-A or destabilization of the polymer hydration process, and wherein the distilled water used as the aqueous medium is pre-degassed by heating to 60-70° C. followed by cooling to ambient temperature or by subjecting the water to vacuum degassing for at least 10 minutes.

7. The process of claim 1, wherein, following Biochanin-A incorporation and prior to sealing, the mixture is allowed to stand undisturbed for a predetermined period of 5-10 minutes to permit natural gravitational settling of transient air pockets introduced during mechanical agitation, and wherein the container is gently tapped to assist bubble migration, and wherein the container used for hydrogel hydration is formed of borosilicate glass possessing low surface reactivity, and wherein the internal walls of the container are optionally coated with a thin film of distilled water prior to transfer of the hydrogel precursor to minimize polymer adhesion, aggregation at the vessel boundaries, or uneven polymer distribution.

8. The process of claim 1, wherein the distilled water medium may optionally be pre-adjusted to a mildly acidic or neutral pH between 5.8 and 7.0 using dilute acid or base to optimize HPMC hydration behavior, and wherein the pH is monitored using a calibrated glass-electrode pH meter prior to polymer addition, with the conditioning improving polymer chain expansion and reducing hydration latency during the stirring process, and wherein the hydrogel precursor is transferred to refrigeration under a controlled cooling rate such that the temperature decreases gradually rather than abruptly, and wherein the cooling profile is achieved by placing the container first at ambient temperature inside the refrigerator door compartment and subsequently moving it deeper into the refrigeration chamber after 2-3 hours.

9. The process of claim 1, wherein the hydrogel precursor, immediately prior to sealing, is subjected to a preliminary macroscopic homogeneity check performed under white light illumination, wherein the operator visually evaluates the dispersion for color uniformity, absence of particulate clusters, consistency of opacity, and smoothness of the polymer phase, and wherein the mixture is re-stirred for a brief period if any visible heterogeneity is detected.

10. The process of claim 1, wherein during the weighing stage the Biochanin-A and hydroxypropyl methyl cellulose are equilibrated to ambient laboratory temperature for at least ten minutes before measurement so that temperature-related mass fluctuations are minimized, and wherein both materials are handled using antistatic weighing paper to avoid electrostatic retention of fine powder during transfer to the mixing vessel, and wherein during the polymer-dispersion stage the distilled water is allowed to rest motionless in the vessel long enough for thermal stabilization before stirring begins, after which the stirring speed is incrementally raised from an initial 150 rpm to a speed in the range of 350-450 rpm, producing a vortex with a depth not greater than one-third of the total liquid depth to allow inward draw of the first portion of hydroxypropyl methyl cellulose.

11. The process of claim 1, wherein during introduction of hydroxypropyl methyl cellulose the powder is dispensed through a narrow-gauge funnel positioned above the fluid vortex so that the polymer contacts the aqueous medium in a dispersed pattern rather than in clumps, and wherein the operator varies the angle of the funnel by small arcs to distribute the powder across the central region of the rotating water column, and wherein the polymer-dispersion stage includes intermittent slowing of the rotational speed for visual assessment of polymer distribution, followed by resumption of higher speeds to promote deeper hydration, and wherein each slowdown event does not exceed ten seconds so that partially hydrated polymer does not settle on the vessel bottom.

12. The process of claim 1, wherein the polymer-dispersion stage is conducted in a vessel having a rounded internal base profile that promotes continuous fluid circulation and restricts the formation of stagnant hydration zones along internal corners, and wherein the vessel is rotated slightly by hand to modify gravitational flow during mixing, and wherein prior to Biochanin-A addition the hydrated polymer is subjected to a brief collapse of the vortex by lifting the stirrer vertically, followed by reinsertion of the stirrer, so that microbubbles introduced during the hydration phase move toward the surface and reduce disruption of the dispersion field during subsequent addition of Biochanin-A.

13. The process of claim 1, wherein during Biochanin-A incorporation each aliquot of Biochanin-A is directed toward a turbulent region of the rotating dispersion located between the center of the vortex and the mid-radius of the vessel so that the powder is drawn into a descending circulation path immediately upon contact with the hydrated polymer, and wherein the Biochanin-A incorporation stage includes temporarily reducing stirring speed by 20-50 rpm during each aliquot addition to promote initial wetting of Biochanin-A particles, followed by restoration of the stirring speed to re-establish the primary mixing regime needed for distribution of Biochanin-A within the polymer matrix.

14. The process of claim 1, wherein during introduction of Biochanin-A a non-metallic spatula is used at intervals to detach adhering aggregates from the vessel walls so that undissolved particulate does not accumulate at boundary layers, and wherein the final homogenization period following Biochanin-A incorporation lasts not less than ten minutes with stirring conducted in a continuous uninterrupted mode to allow each polymer chain segment to interact with dissolved Biochanin-A molecules as the dispersion viscosity increases.

15. The process of claim 1, wherein the transfer stage includes lowering the stirrer speed gradually before switching it off so that the fluid motion decreases smoothly, and wherein the operator waits sufficient time for surface turbulence to dissipate before pouring the mixture into the receiving container, and wherein the container selected for receiving the hydrogel precursor is pre-cleaned by rinsing with distilled water and left to dry under low-turbulence airflow so that environmental particulates do not enter the hydrogel prior to sealing.

16. The process of claim 1, wherein the container interior is optionally moistened with a thin film of distilled water before pouring the hydrogel precursor so that polymer strands contacting the container wall do not undergo premature localized dehydration, wherein after pouring the hydrogel precursor into the container the fluid is left undisturbed for a settling interval of five to ten minutes so that entrained air bubbles migrate toward the surface and leave the bulk material free of internal cavities, wherein sealing of the container is accomplished by wrapping parafilm in multiple overlapping passes across the opening, applying light manual pressure at the rim to achieve close conformation of the sealing material, and extending the sealing material several centimeters down the neck region of the container, and wherein after sealing, the container is positioned initially in the upper region of the refrigeration compartment for gradual temperature descent, and after two to three hours it is relocated to the coldest region inside the refrigeration chamber to support the slow formation of the hydrogel network.

17. The process of claim 1, wherein the refrigeration stage is conducted without any vibration or movement of the container, and wherein the refrigeration shelf selected for storage is cleared of other items to avoid temperature cycling caused by door opening or airflow redirection, wherein the refrigerated hydrogel precursor undergoes progressive viscosity development across the forty-eight-hour storage period, including initial polymer swelling, followed by slow realignment of polymer chains, and culminating in formation of an internally cohesive gel phase, wherein the hydrogel precursor occupies no more than two-thirds of the container volume so that adequate headspace is available for thermal expansion of the aqueous medium during cooling.

18. The process of claim 1, wherein rotational mixing used in the Biochanin-A incorporation stage employs a stirrer blade angle selected to create vertical shear planes in addition to radial shear planes so that distribution of Biochanin-A occurs throughout the full depth of the fluid column, including the lower central region of the vessel where stagnation zones may otherwise occur, wherein the mixing vessel is positioned on a level surface verified by a bubble level so that the vortex axis remains vertical and does not drift laterally, which promotes symmetrical flow of the polymer and Biochanin-A during mixing, and wherein the first portion of HPMC introduced during dispersion is deliberately small, not exceeding 10% of the total polymer mass, so that the initial hydration phase forms a thin polymeric slurry that supports dispersion of the remaining polymer with reduced risk of agglomeration.
